(19) United States
(12) Patent Application Publication
(10) Pub. No.: US 2025/0005761 A1
(43) Pub. Date: Jan. 2, 2025

(54) SYSTEM AND METHOD FOR TOPOLOGICAL CHARACTERIZATION OF TISSUE

(71) Applicant: Spectral MD, Inc., Dallas, TX (US)

(72) Inventors: Jeffrey E. Thatcher, Irving, TX (US); Dominique Meyer, San Diego, CA (US); Peiran Quan, Dallas, TX (US); Faliu Yi, Allen, TX (US)

(21) Appl. No.: 18/766,212

(22) Filed: Jul. 8, 2024

Related U.S. Application Data
(63) Continuation of application No. PCT/US2023/011157, filed on Jan. 19, 2023.
(60) Provisional application No. 63/301,958, filed on Jan. 21, 2022.

(51) Int. Cl. G06T 7/00; G06T 7/11; G06T 7/194; G06T 7/62; G06T 17/20; G16H 30/40; G16H 50/20

(52) U.S. Cl. CPC G06T 7/0016; G06T 7/11; G06T 7/194; G06T 7/62; G06T 7/97; G06T 17/20; G16H 30/40; G16H 50/20; G06T 2200/08; G06T 2207/10152; G06T 2207/30096; G06T 2207/30204; G06T 2210/56

(57) ABSTRACT

An imaging system can include a plurality of imaging sensors configured to receive light reflected by a tissue region in at least a first predetermined waveband and one or more processors configured to cause the image sensors to capture images of the tissue region and identify corresponding sets of pixels in the captured images, each set comprising a pixel of a first image captured by a first image sensor and a pixel of a second image captured by a second image sensor. The processor can determine distance or depth values associated with the individual corresponding sets of pixels based on pixel desparities for the pixels of the corresponding sets of pixels and a baseline distance between the first and second image sensors, and generate a 3D model of the tissue region based at least in part on the distance values.

### CROSS-REFERENCE TO RELATED APPLICATIONS

This application is a continuation of International Application No. PCT/US2023/011157, filed Jan. 19, 2023, entitled SYSTEM AND METHOD FOR TOPOLOGICAL CHARACTERIZATION OF TISSUE, which claims the benefit of U.S. Provisional Application Ser. No. 63/301,958, filed Jan. 21, 2022, entitled SYSTEM AND METHOD FOR TOPOLOGICAL CHARACTERIZATION OF TISSUE, both of which are incorporated by reference herein in their entirety and for all purposes.

### STATEMENT REGARDING FEDERALLY SPONSORED R&D

Some of the work described in this disclosure was made with United States Government support under Contract No. HHSO100201300022C, awarded by the Biomedical Advanced Research and Development Authority (BARDA), within the Office of the Assistant Secretary for Preparedness and Response in the U.S. Department of Health and Human Services. Some of the work described in this disclosure was made with United Government support under Contract Nos. W81XWH-17-C-0170 and/or W81XWH-18-C-0114, awarded by the U.S. Defense Health Agency (DHA). The United States Government may have certain rights in this invention.

### TECHNICAL FIELD

The systems and methods disclosed herein are directed to spectral imaging, and, more particularly, to high precision spectral imaging via a multi-aperture imaging system.

### BACKGROUND

The electromagnetic spectrum is the range of wavelengths or frequencies over which electromagnetic radiation (e.g., light) extends. In order from longer wavelengths to shorter wavelengths, the electromagnetic spectrum includes radio waves, microwaves, infrared (IR) light, visible light (that is, light that is detectable by the structures of the human eye), ultraviolet (UV) light, x-rays, and gamma rays. Spectral imaging refers to a branch of spectroscopy and photography in which some spectral information or a complete spectrum is collected at locations in an image plane. Multispectral imaging systems can capture multiple spectral bands (on the order of a dozen or less and typically at discrete spectral regions), for which spectral band measurements are collected at each pixel, and can refer to bandwidths of about tens of nanometers per spectral channel. Hyperspectral imaging systems measure a greater number of spectral bands, for example as many as 200 or more, with some providing a continuous sampling of narrow bands (e.g., spectral bandwidths on the order of nanometers or less) along a portion of the electromagnetic spectrum.

### SUMMARY

The multispectral imaging systems and techniques disclosed herein have several features, no single one of which is solely responsible for its desirable attributes. Without limiting the scope as expressed by the claims that follow, certain features of the disclosed spectral imaging will now be discussed briefly. One skilled in the art will understand how the features of the disclosed spectral imaging provide several advantages over traditional systems and methods.

In a first aspect, an imaging system comprises a plurality of image sensors configured to receive light reflected by a tissue region in at least a first predetermined waveband; a memory storing computer-executable instructions; and one or more processors configured by the instructions to at least cause each of the at least two image sensors to capture an image of the tissue region; identify corresponding sets of pixels in the captured images, each set comprising a pixel of a first image captured by a first image sensor of the plurality of image sensors and a pixel of a second image captured by a second image sensor of the plurality of image sensors; determine pixel disparities for the pixels of the corresponding sets of pixels, preferably groups of pixel disparities or individual pixel disparities; determine, based at least in part on the pixel disparities a baseline distance between the first and second image sensors, distance values associated with the individual corresponding sets of pixels; and generate a three-dimensional (3D) model of the tissue region based at least in part on the distance values.

In some embodiments, the 3D model of the tissue region comprises at least one of a 3D point cloud, a 3D textured mesh, a 3D parametric surface, or a 3D voxel grid.

In some embodiments, the instructions further configure the one or more processors to determine one or more measurements of at least a portion of the tissue region based at least in part on the 3D model and on a prior calibration of the imaging system. In some embodiments, the one or more measurements comprise at least one of a length measurement, an area measurement, or a volume measurement of a portion of the tissue region. In some embodiments, the one or more measurements comprise a cross-sectional profile of a portion of the tissue region. In some embodiments, the prior calibration is performed based on imaging a standardized calibration target with the imaging system. In some embodiments, the one or more measurements are not determined based on a marker placed within a field of view of the imaging system. In some embodiments, the instructions further configure the one or more processors to perform one or more diagnostic analyses based at least in part on the one or more measurements. In some embodiments, the instructions further configure the one or more processors to store a medical billing code identified based at least in part on the one or more measurements.

In some embodiments, the instructions further configure the one or more processors to segment the captured images prior to identifying the corresponding sets of pixels. In some embodiments, the segmenting comprises identifying at least background pixels and tissue region pixels. In some embodiments, the background pixels are excluded from the identification of corresponding sets of pixels.

In some embodiments, the imaging system further comprises at least one light source configured to illuminate the tissue region with light of at least the first predetermined waveband.

In some embodiments, each image sensor of the plurality of image sensors is configured to image the tissue region in at least the first predetermined waveband and a second waveband different from the second waveband of at least one other image sensor of the plurality of image sensors. In some embodiments, the instructions further configure the one or more processors to obtain multispectral image data associated with the tissue region based on the captured images. In some embodiments, the instructions further configure the one or more processors to align the multispectral image data based on image data corresponding to the first predetermined waveband.

In some embodiments, the tissue region includes at least a portion of a wound, an uneven tissue surface, hyperpigmentation, a lesion, such as a cancerous lesion including, but not limited to, a squamous cell carcinoma, basal cell carcinoma, Merkel cell carcinoma, melanoma, or an actinic keratosis.

In some embodiments, the instructions further configure the one or more processors to detect at least a portion of an edge of the wound, uneven tissue surface, hyperpigmentation, or lesion based on the 3D model. In some embodiments, the instructions further configure the one or more processors to compute one or more gradients of the edges of the wound, uneven tissue surface, hyperpigmentation, or lesion with respect to a surface of the wound, uneven tissue surface, hyperpigmentation, or lesion. In some embodiments, the instructions further configure the one or more processors to determine a status of the wound, uneven tissue surface, hyperpigmentation, or lesion based on a comparison of the one or more gradients to a predetermined gradient threshold. In some embodiments, the instructions further configure the one or more processors to evaluate a change in the status of the wound, uneven tissue surface, hyperpigmentation, or lesion based on a plurality of 3D models generated by the imaging system at different times. In some embodiments, the instructions further configure the one or more processors to determine a characteristic comprising at least one of an area, a rim height, a volume, or a dermal composition of the wound, uneven tissue surface, hyperpigmentation, or lesion based on the 3D model and on the detected at least a portion of an edge of the wound, uneven tissue surface, hyperpigmentation, or lesion. In some embodiments, the instructions further configure the one or more processors to evaluate a change in the characteristic based on a plurality of 3D models generated by the imaging system at different times. In some embodiments, the wound comprises at least one of a burn, a surgical wound, a pressure ulcer, or a diabetic foot ulcer.

In some embodiments, the instructions further configure the one or more processors to perform image rectification or transformation prior to identifying the corresponding sets of pixels. In some embodiments, the image rectification or transformation comprises correcting at least one of scale, distortion, skew, perspective, or rotation of the captured images.

In some embodiments, the instructions further configure the one or more processors to output a visual representation of the tissue region based on the 3D model of the tissue region. In some embodiments, the visual representation comprises two-dimensional image data from at least one of the captured images fitted to the 3D model. In some embodiments, the visual representation comprises a heatmap. In some embodiments, the visual representation comprises a two-dimensional cross-sectional profile of at least a portion of the tissue region.

In some embodiments, the plurality of image sensors include at least three image sensors, and wherein each set further comprises a pixel of a third image captured by a third image sensor of the plurality of image sensors. In some embodiments, the computer-executable instructions further configure the one or more processors to determine confidence values corresponding to the distance values associated with the individual corresponding sets of pixels. In some embodiments, for the individual corresponding sets of pixels, the distance value is determined based on the pixels of the first image and the second image and the confidence value is determined based at least in part on the pixels of the third image. In some embodiments, the confidence value comprises an uncertainty value expressed in a unit of dimension, such as length. In some embodiments, the computer-executable instructions further configure the one or more processors to generate a confidence map of the tissue region based on the 3D model and the determined confidence values. In some embodiments, the computer-executable instructions further configure the one or more processors to exclude from the 3D model pixels having a confidence value satisfying a predetermined threshold. In some embodiments, identifying the corresponding sets of pixels in the captured images comprises evaluating a cost function to identify matching areas or pixels between the first image and the second image. In some embodiments, identifying the matching areas or pixels comprises minimizing the cost function.

In a second aspect, any of the imaging system embodiments described above is used in a wound debridement procedure, optionally before, during or after a wound debridement procedure such as flattening the contour or edge of a wound.

In a third aspect, a method of improving the healing of a wound comprises generating a 3D model of the wound using an imaging system of any one of the preceeding imaging system embodiments and debriding the wound with reference to the 3D model generated by the imaging system such as by referring to the 3D model of the wound prior to, during, or after a debridement procedure, e.g., flattening the contour or edge of the wound.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates an example of light incident on a filter at different chief ray angles.

FIG. 1B is a graph illustrating example transmission efficiencies provided by the filter of FIG. 1A for various chief ray angles.

FIG. 2A illustrates an example of a multispectral image datacube.

FIG. 2B illustrates examples of how certain multispectral imaging technologies generate the datacube of FIG. 2A.

FIG. 2C depicts an example snapshot imaging system that can generate the datacube of FIG. 2A.

FIG. 3A depicts a schematic cross-sectional view of an optical design of an example multi-aperture imaging system with curved multi-bandpass filters, according to the present disclosure.

FIGS. 3B-3D depict example optical designs for optical components of one light path of the multi-aperture imaging system of FIG. 3A.

FIGS. 4A-4E depict an embodiment of a multispectral multi-aperture imaging system, with an optical design as described with respect to FIGS. 3A and 3B.

FIG. 5 depicts another embodiment of a multispectral multi-aperture imaging system, with an optical design as described with respect to FIGS. 3A and 3B.

FIGS. 6A-6C depict another embodiment of a multispectral multi-aperture imaging system, with an optical design as described with respect to FIGS. 3A and 3B.

FIGS. 7A-7B depict another embodiment of a multispectral multi-aperture imaging system, with an optical design as described with respect to FIGS. 3A and 3B.

FIGS. 8A-8B depict another embodiment of a multispectral multi-aperture imaging system, with an optical design as described with respect to FIGS. 3A and 3B.

FIGS. 9A-9C depict another embodiment of a multispectral multi-aperture imaging system, with an optical design as described with respect to FIGS. 3A and 3B.

FIGS. 10A-10B depict another embodiment of a multispectral multi-aperture imaging system, with an optical design as described with respect to FIGS. 3A and 3B.

FIGS. 11A-11B depict an example set of wavebands that can be passed by the filters of the multispectral multi-aperture imaging systems of FIGS. 3A-10B.

FIG. 12 depicts a schematic block diagram of an imaging system that can be used for the multispectral multi-aperture imaging systems of FIGS. 3A-10B.

FIG. 13 is a flowchart of an example process for capturing image data using the multispectral multi-aperture imaging systems of FIGS. 3A-10B.

FIG. 14 depicts a schematic block diagram of a workflow for processing image data, for example image data captured using the process of FIG. 13 and/or using the multispectral multi-aperture imaging systems of FIGS. 3A-10B.

FIG. 15 graphically depicts disparity and disparity correction for processing image data, for example image data captured using the process of FIG. 13 and/or using the multispectral multi-aperture imaging systems of FIGS. 3A-10B.

FIG. 16 graphically depicts a workflow for performing pixel-wise classification on multispectral image data, for example image data captured using the process of FIG. 13, processed according to FIGS. 14 and 15, and/or using the multispectral multi-aperture imaging systems of FIGS. 3A-10B.

FIG. 17 depicts a schematic block diagram of an example distributed computing system including the multispectral multi-aperture imaging systems of FIGS. 3A-10B.

FIGS. 18A-18C illustrate an example handheld embodiment of a multispectral, multi-aperture imaging system.

FIGS. 19A and 19B illustrate an example handheld embodiment of a multispectral, multi-aperture imaging system.

FIGS. 20A and 20B illustrate an example multispectral, multi-aperture imaging system for a small USB 3.0 enclosed in a common camera housing.

FIG. 21 illustrates an example multispectral, multi-aperture imaging system including an additional illuminant for improved image registration.

FIGS. 22A and 22B illustrate an example relationship between camera images of a burn and a generated 3D model.

FIGS. 23A, 23B, and 23C illustrate a unified 3D model of a synthetic leg.

FIG. 24 illustrates an example relationship between pixel disparity and depth or distance information in multi-camera imaging systems.

FIG. 25 illustrates an example relationship between multiple camera views and an estimated 3D tissue scene.

FIGS. 26A-26D illustrate an example process of generating a 3D tissue model based on a plurality of spatially offset 2D images.

FIG. 27A illustrates an example calibration target for keypoint extraction in 3D imaging systems.

FIG. 27B depicts a set of feature correspondence graphs illustrating a calibration estimate of 3D distances between image collection devices around the calibration target of FIG. 27A.

FIG. 27C depicts an example set of camera locations for calibration using the calibration target of FIG. 27A.

FIGS. 28A and 28B illustrate segmentation of tissue pixels from background pixels in an image.

FIGS. 29A-29D illustrate a wound edge geometry assessment using tissue topological characterization of the present technology.

FIG. 30 illustrates wound tracking over a diagnosis and treatment timeline, including a treatment period a, wound state b, feedback component c, and a wound evolution model d.

FIG. 31 illustrates an example Hidden Markov Model (HMM) formulation of a wound evolution model.

FIGS. 32A-32E illustrate an example application of tissue topological characterization techniques of the present technology.

FIG. 33 illustrates an example of a point cloud characterizing a wound generated by the techniques of the present technology.

### DETAILED DESCRIPTION

Generally described, the present disclosure relates to 3D imaging and/or topological characterization of tissue using two or more image sensors. In some aspects, the 3D imaging and/or topological characterization described herein may be performed by a spectral imaging system configured to capture image data in a plurality of different wavebands. Certain systems disclosed herein may be pre-calibrated using one or more targets of known geometry, and the calibration may be used to obtain concrete measurements of dimensions (e.g., lengths) of various aspects of tissue, such as width and/or depth of wounds, ulcers, or any other type of tissue feature. Additional aspects of the present technology will be described in greater detail herein.

In some embodiments, the present disclosure relates to spectral imaging using a multi-aperture system with curved multi-bandpass filters positioned over each aperture. The present disclosure further relates to techniques for implementing spectral unmixing and image registration to generate a spectral datacube using image information received from such imaging systems. The disclosed technology addresses a number of challenges that are typically present in spectral imaging, described below, in order to yield image data that represents precise information about wavelength bands that were reflected from an imaged object. In some embodiments, the systems and methods described herein acquire images from a wide area of tissue (e.g., 5.9×7.9 inches) in a short amount of time (e.g., within 6 seconds or less) and can do so without requiring the injection of imaging contrast agents. In some aspects, for example, the multispectral image system described herein is configured to acquire images from a wide area of tissue, e.g., 5.9×7.9 inches, within 6 seconds or less and, wherein said multispectral image system is also configured to provide tissue analysis information, such as identification of a plurality of burn states, wound states, healing potential, a clinical characteristic including a cancerous or non-cancerous state of the imaged tissue, wound depth, a margin for debridement, or the presence of a diabetic, non-diabetic, or chronic ulcer in the absence of imaging contrast agents. Similarly, in some of the methods described herein, the multispectral image system acquires images from a wide area of tissue, e.g., 5.9×7.9 inches, within 6 seconds or less and said multispectral image system outputs tissue analysis information, such as identification of a plurality of burn states, wound states, healing potential, a clinical characteristic including a cancerous or non-cancerous state of the imaged tissue, wound depth, a margin for debridement, or the presence of a diabetic, non-diabetic, or chronic ulcer in the absence of imaging contrast agents.

One such challenge in existing solutions is that captured images can suffer from color distortions that compromise the quality of the image data. This can be particularly problematic for applications that depend upon precise detection and analysis of certain wavelengths of light using optical filters. Specifically, color shading is a position dependent variation in the wavelength of light across the area of the image sensor, due to the fact that transmittance of a color filter shifts to shorter wavelengths as the angle of light incident on the filter increases. Typically, this effect is observed in interference-based filters, which are manufactured through the deposition of thin layers with varying refractive indices onto a transparent substrate. Accordingly, longer wavelengths (such as red light) can be blocked more at the edges of the image sensor due to larger incident light ray angles, resulting in the same incoming wavelength of light being detected as a spatially non-uniform color across the image sensor. If left uncorrected, color shading manifests as shift in color near the edges of the captured image.

The technology of the present disclosure provides advantages relative to other multi-spectral imaging systems on the market because it is not restrictive in the configuration of lens and/or image sensors and their respective fields of view or aperture sizes. It will be understood that changes to lenses, image sensors, aperture sizes, or other components of the presently disclosed imaging systems may involve other adjustments to the imaging system as would be known to those of ordinary skill in the art. The technology of the present disclosure also provides improvements over other multi-spectral imaging systems in that the components that perform the function of resolving wavelengths or causing the system as a whole to be able to resolve wavelengths (e.g., optical filters or the like) can be separable from the components that transduce light energy into digital outputs (e.g., image sensors or the like). This reduces the cost, complexity, and/or development time to re-configure imaging systems for different multi-spectral wavelengths. The technology of the present disclosure may be more robust than other multi-spectral imaging systems in that it can accomplish the same imaging characteristics as other multi-spectral imaging systems on the market in a smaller and lighter form factor. The technology of the present disclosure is also advantageous relative to other multi-spectral imaging systems in that it can acquire multi-spectral images in a snapshot, video rate, or high speed video rate. The technology of the present disclosure also provides a more robust implementation of multi-spectral imaging systems based on multi-aperture technology as the ability to multiplex several spectral bands into each aperture reduces the number of apertures necessary to acquire any particular number of spectral bands in an imaging data set, thus reducing costs through a reduced number of apertures and improved light collection (e.g., as larger apertures may be used within the fixed size and dimensions of commercially available sensor arrays). Finally, the technology of the present disclosure can provide all of these advantages without a trade-off with respect to resolution or image quality.

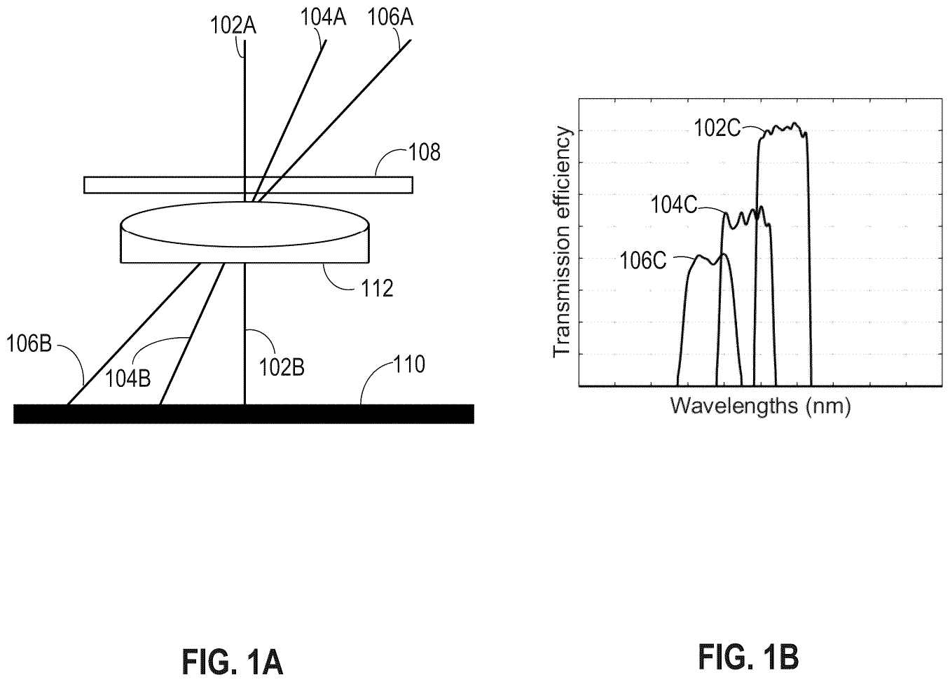

FIG. 1A illustrates an example of a filter 108 positioned along the path of light towards an image sensor 110, and also illustrates light incident on the filter 108 at different ray angles. The rays 102A, 104A, 106A are represented as lines which, after passing through the filter 108, are refracted onto the sensor 110 by a lens 112, which may also be substituted with any other image-forming optics, including but not limited to a mirror and/or an aperture. The light for each ray is presumed in FIG. 1A to be broadband, for example, having a spectral composition extending over a large wavelength range to be selectively filtered by filter 108. The three rays 102A, 104A, 106A each arrive at the filter 108 at a different angle. For illustrative purposes, light ray 102A is shown as being incident substantially normal to filter 108, light ray 104A has a greater angle of incidence than light ray 102A, and light ray 106A has a greater angle of incidence than light ray 104A. The resulting filtered rays 102B, 104B, 106B exhibit a unique spectrum due to the angular dependence of the transmittance properties of the filter 108 as seen by the sensor 110. The effect of this dependence causes a shift in the bandpass of the filter 108 towards shorter wavelengths as the angle of incidence increases. Additionally, the dependence may cause a reduction in the transmission efficiency of the filter 108 and an altering of the spectral shape of the bandpass of the filter 108. These combined effects are referred to as the angular-dependent spectral transmission. FIG. 1B depicts the spectrum of each light ray in FIG. 1A as seen by a hypothetical spectrometer at the location of sensor 110 to illustrate the shifting of the spectral bandpass of filter 108 in response to increasing angle of incidence. The curves 102C, 104C, and 106C demonstrate the shortening of the center wavelength of the bandpass; hence, the shortening of the wavelengths of light passed by the optical system in the example. Also shown, the shape of the bandpass and the peak transmission are altered due to the angle incidence as well. For certain consumer applications, image processing can be applied to remove the visible effects of this angular-dependent spectral transmission. However, these post-processing techniques do not allow for recovery of precise information regarding which wavelength of light was actually incident upon the filter 108. Accordingly, the resulting image data may be unusable for certain high-precision applications.

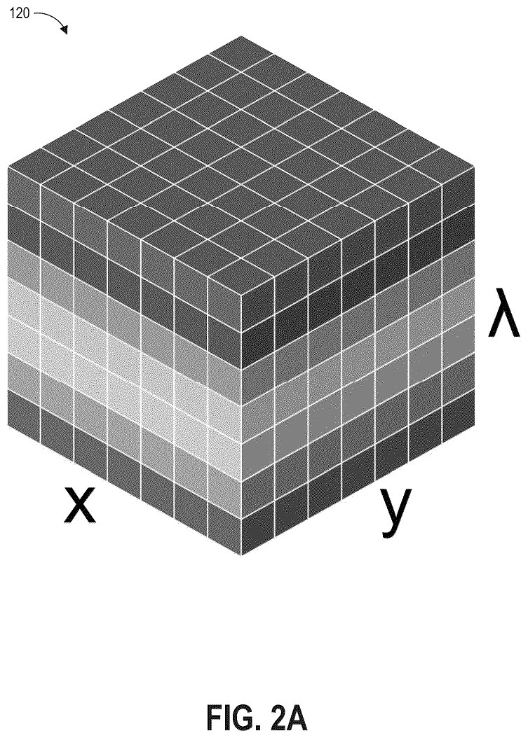

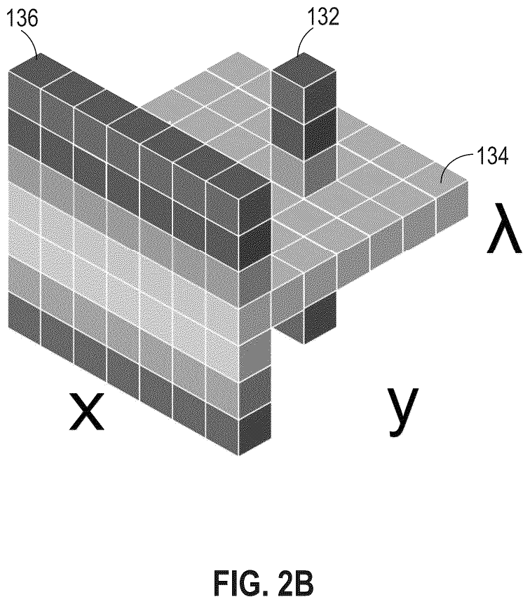

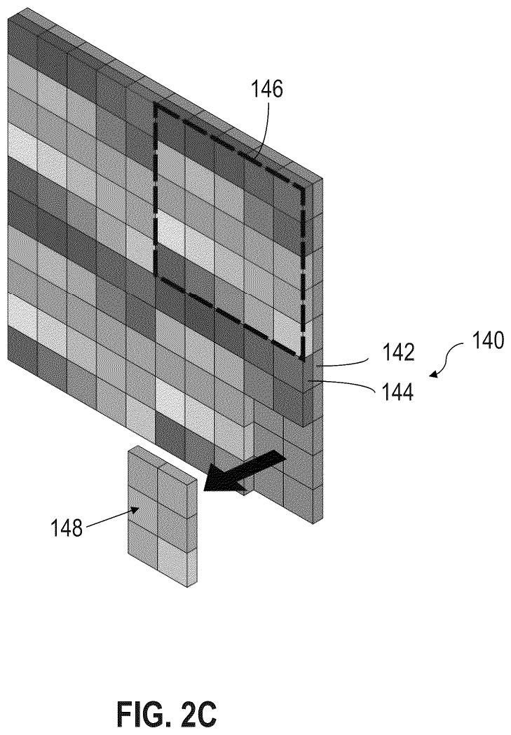

Another challenge faced by certain existing spectral imaging systems is the time required for capture of a complete set of spectral image data, as discussed in connection with FIGS. 2A and 2B. Spectral imaging sensors sample the spectral irradiance I(x,y,λ) of a scene and thus collect a three-dimensional (3D) dataset typically called a datacube. FIG. 2A illustrates an example of a spectral image datacube 120. As illustrated, the datacube 120 represents three dimensions of image data: two spatial dimensions (x and y) corresponding to the two-dimensional (2D) surface of the image sensor, and a spectral dimension (λ) corresponding to a particular wavelength band. The dimensions of the datacube 120 can be given by NₓN_{y}N_{λ}, where Nₓ, N_{y}, and N_{λ} are the number of sample elements along the (x, y) spatial dimensions and spectral axes λ, respectively. Because datacubes are of a higher dimensionality than 2D detector arrays (e.g., image sensors) that are currently available, typical spectral imaging systems either capture time-sequential 2D slices, or planes, of the datacube 120 (referred to herein as “scanning” imaging systems), or simultaneously measure all elements of the datacube by dividing it into multiple 2D elements that can be recombined into datacube 120 in processing (referred to herein as “snapshot” imaging systems).

FIG. 2B illustrates examples of how certain scanning spectral imaging technologies generate the datacube 120. Specifically, FIG. 2B illustrates the portions 132, 134, and 136 of the datacube 120 that can be collected during a single detector integration period. A point scanning spectrometer, for example, can capture a portion 132 that extends across all spectral planes λ at a single (x, y) spatial position. A point scanning spectrometer can be used to build the datacube 120 by performing a number of integrations corresponding to each (x, y) position across the spatial dimensions. A filter wheel imaging system, for example, can capture a portion 134 that extends across the entirety of both spatial dimensions x and y, but only a single spectral plane λ. A wavelength scanning imaging system, such as a filter wheel imaging system, can be used to build the datacube 120 by performing a number of integrations corresponding to the number of spectral planes λ. A line scanning spectrometer, for example, can capture a portion 136 that extends across all spectral dimensions λ and all of one of the spatial dimension (x or y), but only a single point along the other spatial dimension (y or x). A line scanning spectrometer can be used to build the datacube 120 by performing a number of integrations corresponding to each position of this other spatial dimension (y or x).

For applications in which the target object and imaging system are both motionless (or remain relatively still over the exposure times), such scanning imaging systems provide the benefit of yielding a high resolution datacube 120. For line scanning and wavelength scanning imaging systems, this can be due to the fact that each spectral or spatial image is captured using the entire area of the image sensor. However, movement of the imaging system and/or object between exposures can cause artifacts in the resulting image data. For example, the same (x, y) position in the datacube 120 can actually represent a different physical location on the imaged object across the spectral dimension λ. This can lead to errors in downstream analysis and/or impose an additional requirement for performing registration (e.g., aligning the spectral dimension λ so that a particular (x, y) position corresponds to the same physical location on the object).

In comparison, a snapshot imaging system 140 can capture an entire datacube 120 in a single integration period or exposure, thereby avoiding such motion-induced image quality issues. FIG. 2C depicts an example image sensor 142 and an optical filter array such as a color filter array (CFA) 144 that can be used to create a snapshot imaging system. The CFA 144 in this example is a repeating pattern of color filter units 146 across the surface of the image sensor 142. This method of acquiring spectral information can also be referred to as a multispectral filter array (MSFA) or a spectrally resolved detector array (SRDA). In the illustrated example, the color filter unit 146 includes a 5×5 arrangement of different color filters, which would generate 25 spectral channels in the resulting image data. By way of these different color filters, the CFA can split incoming light into the bands of the filters, and direct the split light to dedicated photoreceptors on the image sensor. In this way, for a given color 148, only 1/25^{th} of the photoreceptors actually detect a signal represent light of that wavelength. Thus, although 25 different color channels can be generated in a single exposure with this snapshot imaging system 140, each color channel represents a smaller quantity of measured data than the total output of the sensor 142. In some embodiments, a CFA may include one or more of a filter array (MSFA), a spectrally resolved detector array (SRDA), and/or may include a conventional Bayer filter, CMYK filter, or any other absorption-based or interference-based filters. One type of interference based filter would be an array of thin film filters arranged in a grid with each element of the grid corresponding to one or more sensor elements. Another type of interference based filter is a Fabry-Pérot filter. Nanoetched interference Fabry-Pérot filters, which exhibit typical bandpass full-width-at-half-maxima (FWHM) on the order of 20 to 50 nm, are advantageous because they can be used in some embodiments due to the slow roll-off of the filters' passband seen in the transition from its center wavelength to its blocking band. These filters also exhibit a low OD in these blocking bands further enabling increased sensitivity to light outside of their passbands. These combined effects makes these specific filters sensitive to spectral regions that would otherwise be blocked by the fast roll-off of a high OD interference filter with a similar FWHM made with many thin film layers in a coating deposition process such as in evaporative deposition or in ion-beam sputtering. In embodiments with dye-based CMYK or RGB (Bayer) filter configurations, the slow spectral roll-off and the large FWHM of individual filter passbands are preferred and provide a unique spectral transmission percentage to individual wavelengths throughout an observed spectrum.

Accordingly, the datacube 120 that results from a snapshot imaging system will have one of two properties that can be problematic for precision imaging applications. As a first option, the datacube 120 that results from a snapshot imaging system can have smaller Nₓ and N_{y} sizes than the (x, y) size of the detector array and, thus be of lower resolution than the datacube 120, which would be generated by a scanning imaging system having the same image sensor. As a second option, the datacube 120 that results from a snapshot imaging system can have the same Nₓ and N_{y} sizes as the (x, y) size of the detector array due to interpolating values for certain (x, y) positions. However, the interpolation used to generate such a datacube means that certain values in the datacube are not actual measurements of the wavelength of light incident on the sensor, but rather estimates of what the actual measurement may be based on surrounding values.

Another existing option for single-exposure multispectral imaging is the multispectral beamsplitter. In such imaging systems, beamsplitter cubes split incident light into distinct color bands, with each band observed by independent image sensors. While one can change the beamsplitter designs to adjust the measured spectral bands, it is not easy to divide the incident light into more than four beams without compromising the system performance. Thus, four spectral channels appear to be the practical limit of this approach. A closely related method is to use thin-film filters instead of the bulkier beamsplitter cubes/prisms to split the light, however this approach is still limited to about six spectral channels due to space limitations and cumulative transmission losses through successive filters.

The aforementioned problems, among others, are addressed in some embodiments by the disclosed multi-aperture spectral imaging system with curved multi-bandpass filters to filter light incoming through each aperture, and the associated image data processing techniques. This particular configuration is able to achieve all of the design goals of fast imaging speeds, high resolution images, and precise fidelity of detected wavelengths. Accordingly, the disclosed optical design and associated image data processing techniques can be used in portable spectral imaging systems and/or to image moving targets, while still yielding a datacube suitable for high precision applications (e.g., clinical tissue analysis, biometric recognition, transient clinical events). These higher precision applications may include the diagnosis of melanoma in the preceeding stages (0 through 3) before metastasis, the classification of burn wound severity on skin tissue, or the tissue diagnosis of diabetic foot ulcer severity. Accordingly, the small form factor and the snapshot spectral acquisition as depicted in some embodiments will enable the use of this invention in clinical environments with transient events, which include the diagnosis of several different retinopathies (e.g. non proliferative diabetic retinopathy, proliferative diabetic retinopathy, and age-related macular degeneration) and the imaging of moving pediatric patients. Accordingly, it will be appreciated by one of skill in the art that the use of a multi-aperture system with flat or curved multi-bandpass filters, as disclosed herein, represents a significant technological advance over prior spectral imaging implementations. Specifically, the multi-aperture system may enable the collection of 3D spatial images of or relating to object curvature, depth, volume, and/or area based on the calculated disparity of the perspective differences between each aperture. However, the multi-aperture strategies presented here are not limited to any specific filter and may include flat and/or thin filters, based on either interference or absorptive filtering. This invention, as disclosed herein, can be modified to include flat filters in the image space of the imaging system in the event of suitable lenses or apertures that use a small or acceptable range of incidence angles. Filters may also be placed at the aperture stop or at the entrance/exit pupil of the imaging lenses as one skilled in the art of optical engineering may see fit to do so.

Various aspects of the disclosure will now be described with regard to certain examples and embodiments, which are intended to illustrate but not limit the disclosure. Although the examples and embodiments described herein will focus, for the purpose of illustration, on specific calculations and algorithms, one of skill in the art will appreciate the examples are to illustrate only, and are not intended to be limiting. For example, although some examples are presented in the context of a multispectral imaging, the disclosed multi-aperture imaging system and associated filters can be configured to achieve hyperspectral imaging in other implementations. Further, although certain examples are presented as achieving benefits for handheld and/or moving target applications, it will be appreciated that the disclosed imaging system design and associated processing techniques can yield a high precision datacube suitable for fixed imaging systems and/or for analysis of relatively motionless targets.

### Overview of Electromagnetic Ranges and Image Sensors

Certain colors or portions of the electromagnetic spectrum are referred to herein, and will now be discussed with respect to their wavelength as defined by the ISO 21348 definitions of irradiance spectral categories. As described further below, in certain imaging applications the wavelength ranges for specific colors can be grouped together to pass through a certain filter.

Electromagnetic radiation ranging from wavelengths of or approximately 760 nm to wavelengths of or approximately 380 nm are typically considered the “visible” spectrum, that is, the portion of the spectrum recognizable by the color receptors of the human eye. Within the visible spectrum, red light typically is considered to have a wavelength of or approximately 700 nanometers (nm), or to be in the range of or approximately 760 nm to 610 nm or approximately 610 nm. Orange light typically is considered to have a wavelength of or approximately 600 nm, or to be in the range of or approximately 610 nm to approximately 591 nm or 591 nm. Yellow light typically is considered to have a wavelength of or approximately 580 nm, or to be in the range of or approximately 591 nm to approximately 570 nm or 570 nm. Green light typically is considered to have a wavelength of or approximately 550 nm, or to be in the range of or approximately 570 nm to approximately 500 nm or 500 nm. Blue light typically is considered to have a wavelength of or approximately 475 nm, or to be in the range of or approximately 500 nm to approximately 450 nm or 450 nm. Violet (purple) light typically is considered to have a wavelength of or approximately 400 nm, or to be in the range of or approximately 450 nm to approximately 360 nm or 360 nm.

Turning to ranges outside of the visible spectrum, infrared (IR) refers to electromagnetic radiation with longer wavelengths than those of visible light, and is generally invisible to the human eye. IR wavelengths extend from the nominal red edge of the visible spectrum at approximately 760 nm or 760 nm to approximately 1 millimeter (mm) or 1 mm. Within this range, near infrared (NIR) refers to the portion of the spectrum that is adjacent to the red range, ranging from wavelengths between approximately 760 nm or 760 nm to approximately 1400 nm or 1400 nm.

Ultraviolet (UV) radiation refers to some electromagnetic radiation with shorter wavelengths than those of visible light, and is generally invisible to the human eye. UV wavelengths extend from the nominal violet edge of the visible spectrum at approximately 40 nm or 40 nm to approximately 400 nm. Within this range, near ultraviolet (NUV) refers to the portion of the spectrum that is adjacent to the violet range, ranging from wavelengths between approximately 400 nm or 400 nm to approximately 300 nm or 300 nm, middle ultraviolet (MUV) ranges from wavelengths between approximately 300 nm or 300 nm to approximately 200 nm or 200 nm, and far ultraviolet (FUV) ranges from wavelengths between approximately 200 nm or 200 nm to approximately 122 nm or 122 nm.

The image sensors described herein can be configured to detect electromagnetic radiation in any of the above-described ranges, depending upon the particular wavelength ranges that are suitable for a particular application. The spectral sensitivity of a typical silicon-based charge-coupled device (CCD) or complementary metal-oxide-semiconductor (CMOS) sensor extends across the visible spectrum, and also extends considerably into the near-infrared (IR) spectrum and sometimes into the UV spectrum. Some implementations can alternatively or additionally use back-illuminated or front-illuminated CCD or CMOS arrays. For applications requiring high SNR and scientific-grade measurements, some implementations can alternatively or additionally use either scientific complementary metal-oxide-semiconductor (sCMOS) cameras or electron multiplying CCD cameras (EMCCD). Other implementations can alternatively or additionally use sensors known to operate in specific color ranges (e.g., short-wave infrared (SWIR), mid-wave infrared (MWIR), or long-wave infrared (LWIR)) and corresponding optical filter arryas, based on the intended applications. These may alternatively or additionally include cameras based around detector materials including indium gallium arsenide (InGaAs) or indium antimonide (InSb) or based around microbolometer arrays.

The image sensors used in the disclosed multispectral imaging techniques may be used in conjunction with an optical filter array such as a color filter array (CFA). Some CFAs can split incoming light in the visible range into red (R), green (G), and blue (B) categories to direct the split visible light to dedicated red, green, or blue photodiode receptors on the image sensor. A common example for a CFA is the Bayer pattern, which is a specific pattern for arranging RGB color filters on a rectangular grid of photosensors. The Bayer pattern is 50% green, 25% red and 25% blue with rows of repeating red and green color filters alternating with rows of repeating blue and green color filters. Some CFAs (e.g., for RGB-NIR sensors) can also separate out the NIR light and direct the split NIR light to dedicated photodiode receptors on the image sensor.

As such, the wavelength ranges of the filter components of the CFA can determine the wavelength ranges represented by each image channel in a captured image. Accordingly, a red channel of an image may correspond to the red wavelength regions of the color filter and can include some yellow and orange light, ranging from approximately 570 nm or 570 nm to approximately 760 nm or 760 nm in various embodiments. A green channel of an image may correspond to a green wavelength region of a color filter and can include some yellow light, ranging from approximately 570 nm or 570 nm to approximately 480 nm or 480 nm in various embodiments. A blue channel of an image may correspond to a blue wavelength region of a color filter and can include some violet light, ranging from approximately 490 nm or 490 nm to approximately 400 nm or 400 nm in various embodiments. As a person of ordinary skill in the art will appreciate, exact beginning and ending wavelengths (or portions of the electromagnetic spectrum) that define colors of a CFA (for example, red, green, and blue) can vary depending upon the CFA implementation.

Further, typical visible light CFAs are transparent to light outside the visible spectrum. Therefore, in many image sensors the IR sensitivity is limited by a thin-film reflective IR filter at the face of the sensor that blocks the infrared wavelength while passing visible light. However, this may be omitted in some of the disclosed imaging systems to allow of passage of IR light. Thus, the red, green, and/or blue channels may also be used to collect IR wavelength bands. In some implementations the blue channel may also be used to collect certain NUV wavelength bands. The distinct spectral responses of the red, green, and blue channels with regard to their unique transmission efficiencies at each wavelength in a spectral image stack may provide a uniquely weighted response of spectral bands to be unmixed using the known transmission profiles. For example, this may include the known transmission response in IR and UV wavelength regions for the red, blue, and green channels, enabling their use in the collection of bands from these regions.

As described in further detail below, additional color filters can be placed before the CFA along the path of light towards the image sensor in order to selectively refine the specific bands of light that become incident on the image sensor. Some of the disclosed filters can be either a combination of dichroic (thin-film) and/or absorptive filters or a single dichroic and/or absorptive filter. Some of the disclosed color filters can be bandpass filters that pass frequencies within a certain range (in a passband) and reject (attenuates) frequencies outside that range (in a blocking range). Some of the disclosed color filters can be multi-bandpass filters that pass multiple discontinuous ranges of wavelengths. These “wavebands” can have smaller passband ranges, larger blocking range attenuation, and sharper spectral roll-off, which is defined as the steepness of the spectral response as the filter transitions from the passband to the blocking range, than the larger color range of the CFA filter. For example, these disclosed color filters can cover a passband of approximately 20 nm or 20 nm or approximately 40 nm or 40 nm. The particular configuration of such color filters can determine the actual wavelength bands that are incident upon the sensor, which can increase the precision of the disclosed imaging techniques. The color filters described herein can be configured to selectively block or pass specific bands of electromagnetic radiation in any of the above-described ranges, depending upon the particular wavelength bands that are suitable for a particular application.

As described herein, a “pixel” can be used to describe the output generated by an element of the 2D detector array. In comparison, a photodiode, a single photosensitive element in this array, behaves as a transducer capable of converting photons into electrons via the photoelectric effect, which is then in turn converted into a usable signal used to determine the pixel value. A single element of the datacube can be referred to as a “voxel” (e.g., a volume element). A “spectral vector” refers to a vector describing the spectral data at a particular (x, y) position in a datacube (e.g., the spectrum of light received from a particular point in the object space). A single horizontal plane of the datacube (e.g., an image representing a single spectral dimension), is referred to herein as a an “image channel”. Certain embodiments described herein may capture spectral video information, and the resulting data dimensions can assume the “hypercube” form Nₓ N_{y}N_{λ}Nₜ, where Nₜ is the number of frames captured during a video sequence.

Overview of Example Multi-Aperture Imaging Systems with Curved Multi-Bandpass Filters

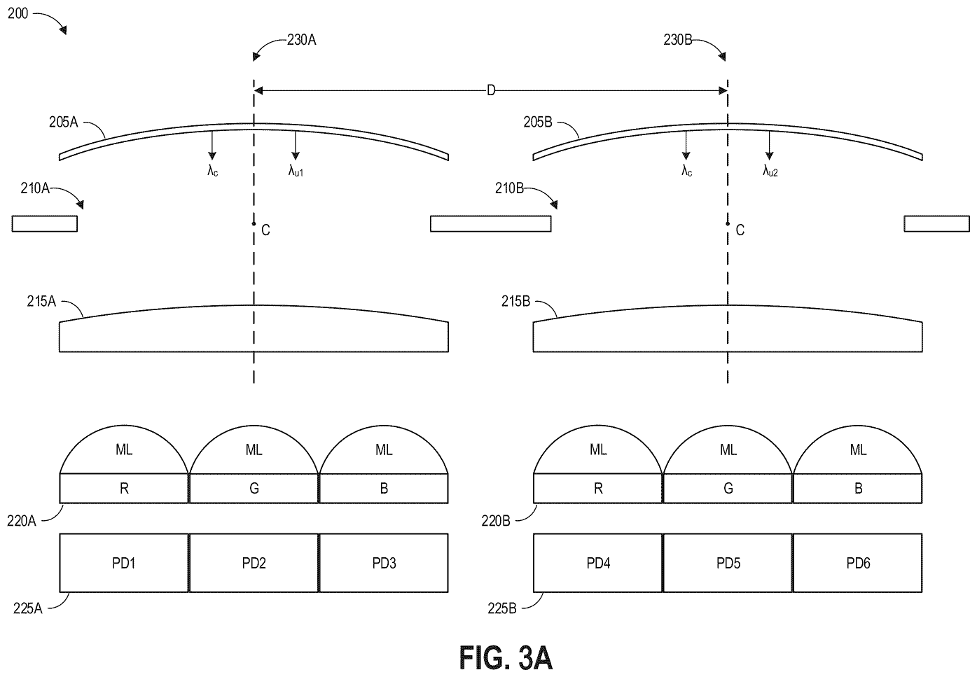

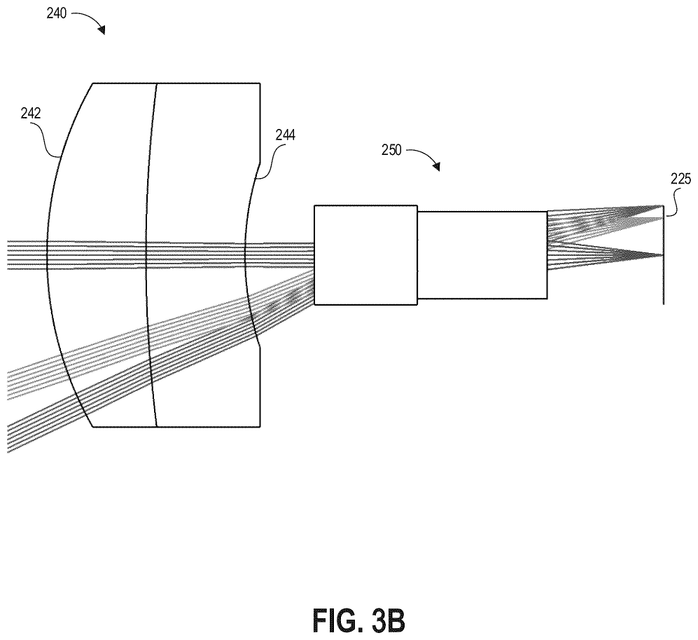

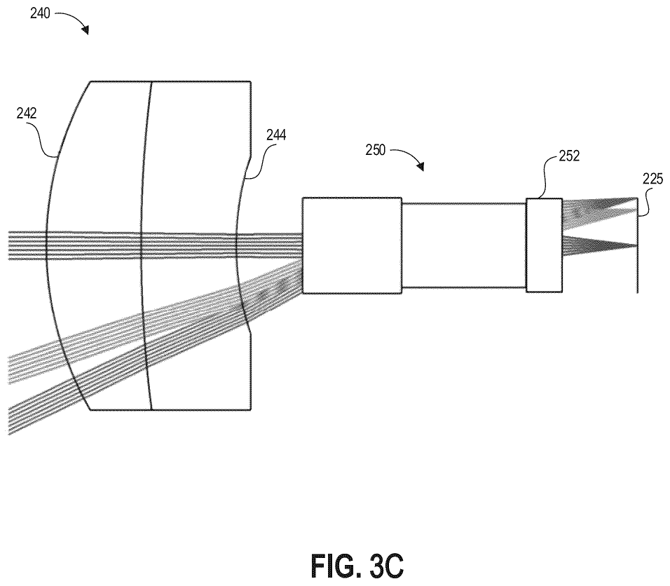

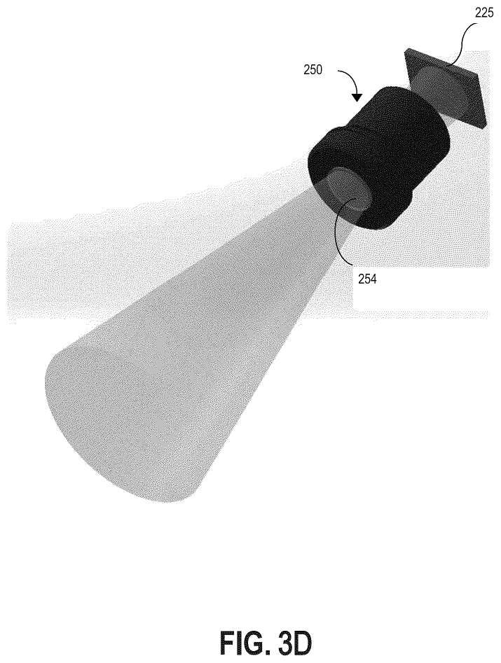

FIG. 3A depicts a schematic view of an example multi-aperture imaging system 200 with curved multi-bandpass filters, according to the present disclosure. The illustrated view includes a first image sensor region 225A (photodiodes PD1-PD3) and a second image sensor region 225B (photodiodes PD4-PD6). The photodiodes PD1-PD6 can be, for example, photodiodes formed in a semiconductor substrate, for example in a CMOS image sensor. Generally, each of the photodiodes PD1-PD6 can be a single unit of any material, semiconductor, sensor element or other device that converts incident light into current. It will be appreciated that a small portion of the overall system is illustrated for the purpose of explaining its structure and operation, and that in implementation image sensor regions can have hundreds or thousands of photodiodes (and corresponding color filters). The image sensor regions 225A and 225B may be implemented as separate sensors, or as separate regions of the same image sensor, depending upon the implementation. Although FIG. 3A depicts two apertures and corresponding light paths and sensor regions, it will be appreciated that the optical design principles illustrated by FIG. 3A can be extended to three or more apertures and corresponding light paths and sensor regions, depending upon the implementation.

The multi-aperture imaging system 200 includes a first opening 210A that provides a first light path towards the first sensor region 225A, and a second opening 210B that provides a first light path towards the second sensor region 225B. These apertures may be adjustable to increase or decrease the brightness of the light that falls on the image, or so that the duration of particular image exposures can be changed and the brightness of the light that falls on the image sensor regions does not change. These apertures may also be located at any position along the optical axes of this multi-aperture system as deemed reasonable by one skilled in the art of optical design. The optical axis of the optical components positioned along the first light path is illustrated by dashed line 230A and the optical axis of the optical components positioned along the second light path is illustrated by dashed line 230B, and it will be appreciated that these dashed lines do not represent a physical structure of the multi-aperture imaging system 200. The optical axes 230A, 230B are separated by a distance D, which can result in disparity between the images captured by the first and second sensor regions 225A, 225B. Disparity refers to the distance between two corresponding points in the left and right (or upper and lower) images of a stereoscopic pair, such that the same physical point in the object space can appear in different locations in each image. Processing techniques to compensate for and leverage this disparity are described in further detail below.

Each optical axis 230A, 230B passes through a center C of the corresponding aperture, and the optical components can also be centered along these optical axes (e.g., the point of rotational symmetry of an optical component can be positioned along the optical axis). For example, the first curved multi-bandpass filter 205A and first imaging lens 215A can be centered along the first optical axis 230A, and the second curved multi-bandpass filter 205B and second imaging lens 215B can be centered along the second optical axis 230B.

As used herein with respect to positioning of optical elements, “over” and “above” refer to the position of a structure (for example, a color filter or lens) such that light entering the imaging system 200 from the object space propagates through the structure before it reaches (or is incident upon) another structure. To illustrate, along the first light path, the curved multi-bandpass filter 205A is positioned above the aperture 210A, the aperture 210A is positioned above imaging lens 215A, the imaging lens 215A is positioned above the CFA 220A, and the CFA 220A is positioned above the first image sensor region 225A. Accordingly, light from the object space (e.g., the physical space being imaged) first passes through the curved multi-bandpass filter 205A, then the aperture 210A, then the imaging lens 215A, then the CFA 220A, and finally is incident on the first image sensor region 225A. The second light path (e.g., curved multi-bandpass filter 205B, aperture 210B, imaging lens 215B, CFA 220B, second image sensor region 225B) follows a similar arrangement. In other implementations, the aperture 210A, 210B and/or imaging lenses 215A, 215B can be positioned above the curved multi-bandpass filter 205A, 205B. Additionally, other implementations may not use a physical aperture and may rely on the clear aperture of the optics to control the brightness of light that is imaged onto the sensor region 225A, 225B. Accordingly, the lens 215A, 215B may be placed above the aperture 210A, 210B and curved multi-bandpass filter 205A, 205B. In this implementation, the aperture 210A, 210B and lens 215A, 215B may be also be placed over or under each other as deemed necessary by one skilled in the art of optical design.

The first CFA 220A positioned over the first sensor region 225A and the second CFA 220B positioned over the second sensor region 225B can act as wavelength-selective pass filters and split incoming light in the visible range into red, green, and blue ranges (as indicated by the R, G, and B notation). The light is “split” by allowing only certain selected wavelengths to pass through each of the color filters in the first and second CFAs 220A, 220B. The split light is received by dedicated red, green, or blue diodes on the image sensor. Although red, blue, and green color filters are commonly used, in other embodiments the color filters can vary according to the color channel requirements of the captured image data, for example including ultraviolet, infrared, or near-infrared pass filters, as with an RGB-IR CFA.

As illustrated, each filter of the CFA is positioned over a single photodiode PD1-PD6. FIG. 3A also illustrates example microlenses (denoted by ML) that can be formed on or otherwise positioned over each color filter, in order to focus incoming light onto active detector regions. Other implementations may have multiple photodiodes under a single filter (e.g., clusters of 2, 4, or more adjacent photodiodes). In the illustrated example, photodiode PD1 and photodiode PD4 are under red color filters and thus would output red channel pixel information; photodiode PD2 and photodiode PD5 are under green color filters and, thus would output green channel pixel information; and photodiode PD3 and photodiode PD6 are under blue color filters and thus would output blue channel pixel information. Further, as described in more detail below, the specific color channels output by given photodiodes can be further limited to narrower wavebands based on activated illuminants and/or the specific wavebands passed by the multi-bandpass filters 205A, 205B, such that a given photodiode can output different image channel information during different exposures.

The imaging lenses 215A, 215B can be shaped to focus an image of the object scene onto the sensor regions 225A, 225B. Each imaging lens 215A, 215B may be composed of as many optical elements and surfaces needed for image formation and are not limited to single convex lenses as presented in FIG. 3A, enabling the use of a wide variety of imaging lenses or lens assemblies that would be available commercially or by custom design. Each element or lens assembly may be formed or bonded together in a stack or held in series using an optomechanical barrel with a retaining ring or bezel. In some embodiments, elements or lens assemblies may include one or more bonded lens groups, such as two or more optical components cemented or otherwise bonded together. In various embodiments, any of the multi-bandpass filters described herein may be positioned in front of a lens assembly of the multispectral image system, in front of a singlet of the multispectral image system, behind a lens assembly of the multispectral image system, behind a singlet of the multispectral image system, inside a lens assembly of the multispectral image system, inside a bonded lens group of the multispectral image system, directly onto a surface of a singlet of the multispectral image system, or directly onto a surface of an element of a lens assembly of the multispectral image system. Further, the aperture 210A and 210B may be removed, and the lenses 215A, 215B may be of the variety typically used in photography with either digital-single-lens-reflex (DSLR) or mirrorless cameras. Additionally, these lenses may be of the variety used in machine vision using C-mount or S-mount threading for mounting. Focus adjustment can be provided by movement of the imaging lenses 215A, 215B relative to the sensor regions 225A, 225B or movement of the sensor regions 225A, 225B relative to the imaging lenses 215A, 215B, for example based on manual focusing, contrast-based autofocus, or other suitable autofocus techniques.

The multi-bandpass filters 205A, 205B can be each configured to selectively pass multiple narrow wavebands of light, for example wavebands of 10-50 nm in some embodiments (or wider or narrower wavebands in other embodiments). As illustrated in FIG. 3A, both multi-bandpass filters 205A, 205B can pass waveband λ_{c} (the “common waveband”). In implementations with three or more light paths, each multi-bandpass filter can pass this common waveband. In this manner, each sensor region captures image information at the same waveband (the “common channel”). This image information in this common channel can be used to register the sets of images captured by each sensor region, as described in further detail below. Some implementations may have one common waveband and corresponding common channel, or may have multiple common wavebands and corresponding common channels.

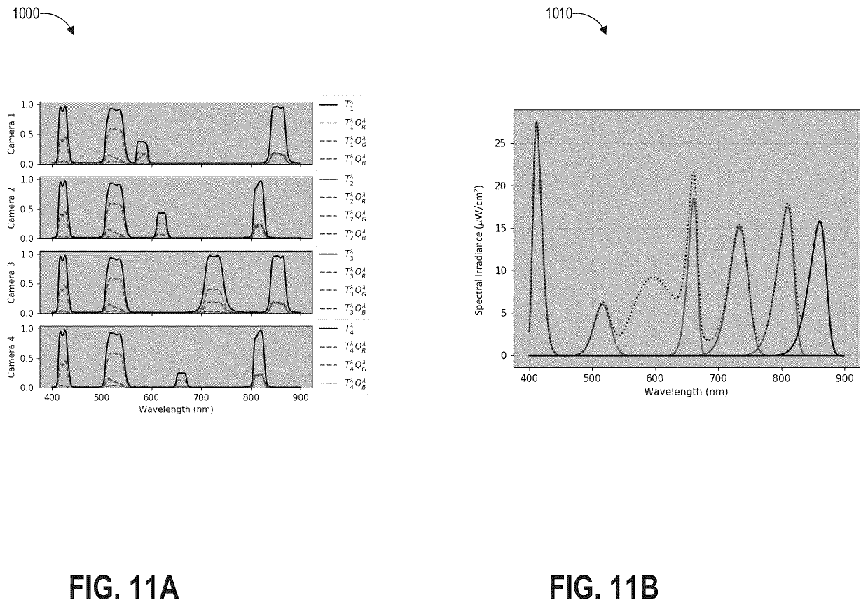

In addition to the common waveband 2, each multi-bandpass filters 205A, 205B can be each configured to selectively pass one or more unique wavebands. In this manner, the imaging system 200 is able to increase the number of distinct spectral channels captured collectively by the sensor regions 205A, 205B beyond what can be captured by a single sensor region. This is illustrated in FIG. 3A by multi-bandpass filters 205A passing unique waveband λᵤₗ, and multi-bandpass filters 205B passing unique waveband λᵤ₂, where λᵤₗ and λᵤ₂ represent different wavebands from one another. Although depicted as passing two wavebands, the disclosed multi-bandpass can each pass a set of two or more wavebands. For example, some implementations can pass four wavebands each, as described with respect to FIGS. 11A and 11B. In various embodiments, a larger number of wavebands may be passed. For example, some four-camera implementations may include multi-bandpass filters configured to pass 8 wavebands. In some embodiments, the number of wavebands may be, for example, 4, 5, 6, 7, 8, 9, 10, 12, 15, 16, or more wavebands.

The multi-bandpass filters 205A, 205B have a curvature selected to reduce the angular-dependent spectral transmission across the respective sensor regions 225A, 225B. As a result, when receiving narrowband illumination from the object space, each photodiode across the area of the sensor regions 225A, 225B that is sensitive to that wavelength (e.g., the overlying color filter passes that wavelength) should receive substantially the same wavelength of light, rather than photodiodes near the edge of the sensor experiencing the wavelength shift described above with respect to FIG. 1A. This can generate more precise spectral image data than using flat filters.

FIG. 3B depicts an example optical design for optical components of one light path of the multi-aperture imaging system of FIG. 3A. Specifically, FIG. 3B depicts a custom achromatic doublet 240 that can be used to provide the multi-bandpass filters 205A, 205B. The custom achromatic doublet 240 passes light through a housing 250 to an image sensor 225. The housing 250 can include openings 210A, 210B and imaging lens 215A, 215B described above.

The achromatic doublet 240 is configured to correct for optical abberations as introduced by the incorporation of surfaces required for the multi-bandpass filter coatings 205A, 205B. The illustrated achromatic doublet 240 includes two individual lenses, which can be made from glasses or other optical materials having different amounts of dispersion and different refractive indices. Other implementations may use three or more lenses. These achromatic doublet lenses can be designed to incorporate the multi-bandpass filter coatings 205A, 205B on the curved front surface 242 while eliminating optical aberrations introduced that would otherwise be present through the incorporation of a curved singlet optical surface with the deposited filter coatings 205A, 205B while still limiting optical or focusing power provided by the achromatic doublet 240 due to the combinatorial effect of the curved front surface 242 and the curved back surface of 244 while still keeping the primary elements for focusing light restricted to the lenses housed in housing 250. Thus, the achromatic doublet 240 can contribute to the high precision of image data captured by the system 200. These individual lenses can be mounted next to each other, for example being bonded or cemented together, and shaped such that the aberration of one of the lenses is counterbalanced by that of the other. The achromatic doublet 240 curved front surface 242 or the curved back surface 244 can be coated with the multi-bandpass filter coating 205A, 205B. Other doublet designs may be implemented with the systems described herein.

Further variations of the optical designs described herein may be implemented. For example, in some embodiments a light path may include a singlet or other optical singlet such as of the positive or negative meniscus variety as depicted in FIG. 3A instead of the doublet 240 depicted in FIG. 3B. FIG. 3C illustrates an example implementation in which a flat filter 252 is included between the lens housing 250 and the sensor 225. The achromatic doublet 240 in FIG. 3C provides optical aberration correction as introduced by the inclusion of the flat filter 252 containing a multi-bandpass transmission profile while not significantly contributing to the optical power as provided by the lenses contained in housing 250. FIG. 3D illustrates another example of an implementation in which the multi-bandpass coating is implemented by means of a multi-bandpass coating 254 applied to the front surface of the lens assembly contained within the housing 250. As such, this multi-bandpass coating 254 may be applied to any curved surface of any optical element residing within housing 250.

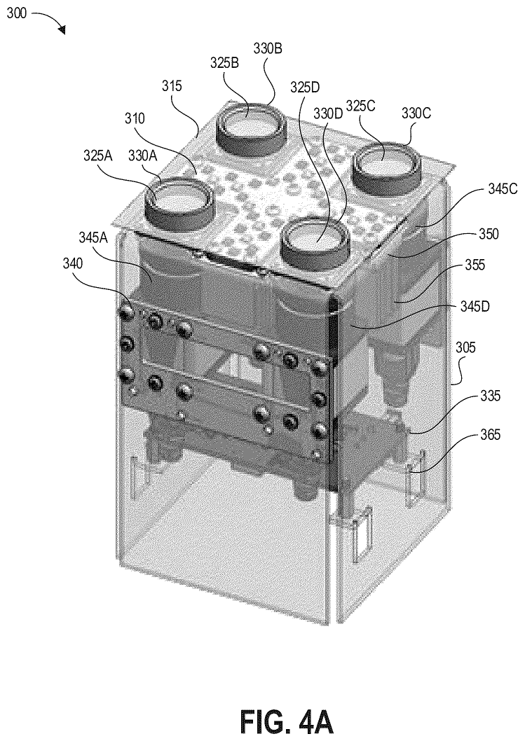

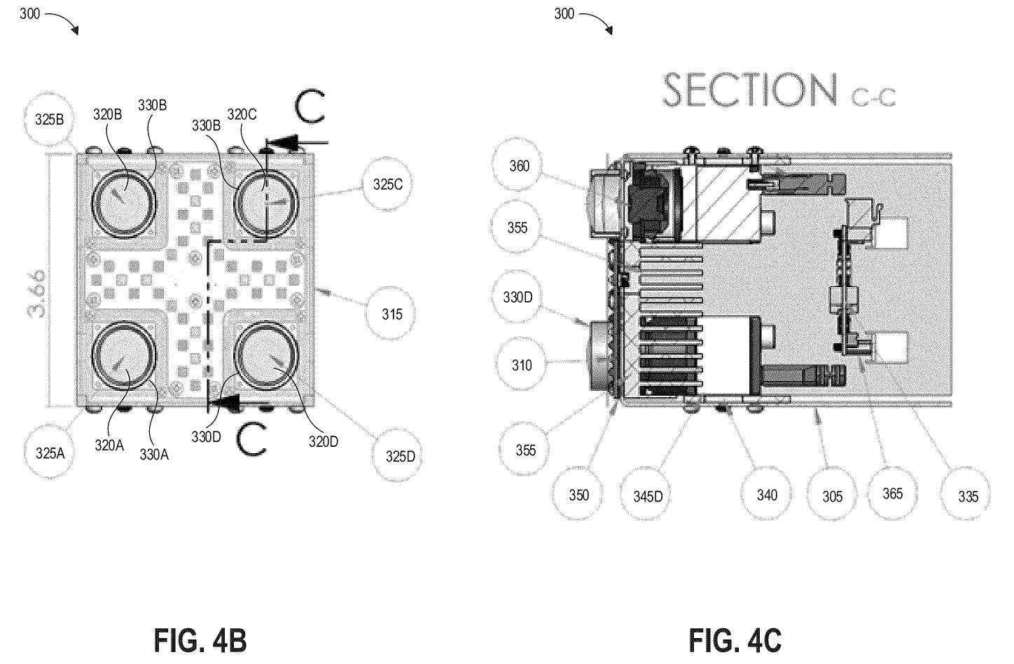

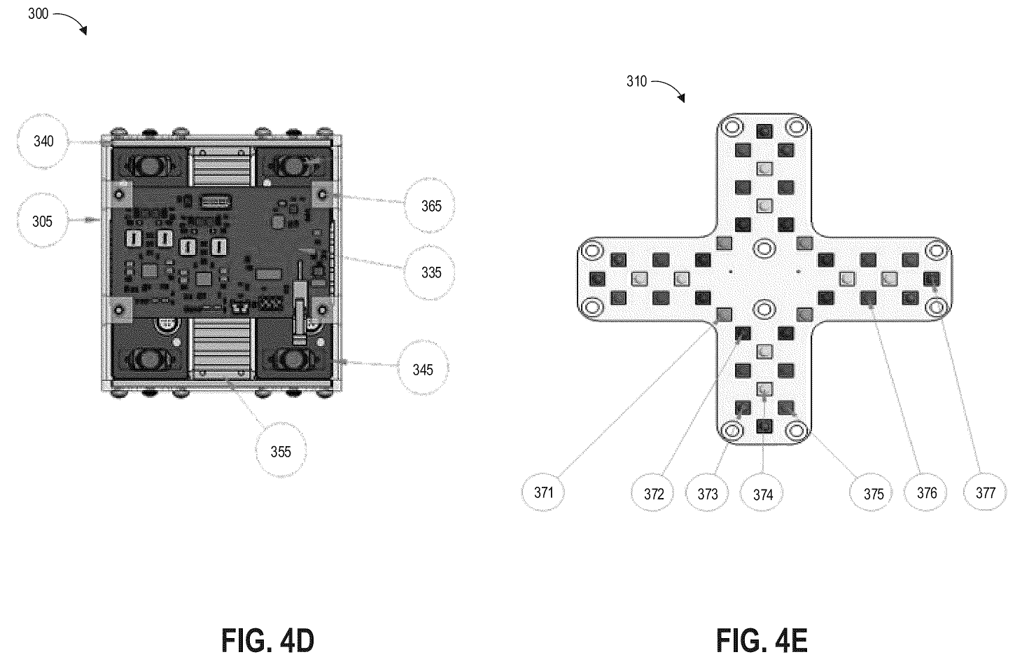

FIGS. 4A-4E depict an embodiment of a multispectral, multi-aperture imaging system 300, with an optical design as described with respect to FIGS. 3A and 3B. Specifically, FIG. 4A depicts a perspective view of the imaging system 300 with the housing 305 illustrated with translucency to reveal interior components. The housing 305 may be larger or smaller relative to the illustrated housing 305, for example, based on a desired amount of embedded computing resources. FIG. 4B depicts a front view of the imaging system 300. FIG. 4C depicts a cutaway side view of the imaging system 300, cut along line C-C illustrated in FIG. 4B. FIG. 4D depicts a bottom view of the imaging system 300 depicting the processing board 335. FIGS. 4A-4D are described together below.

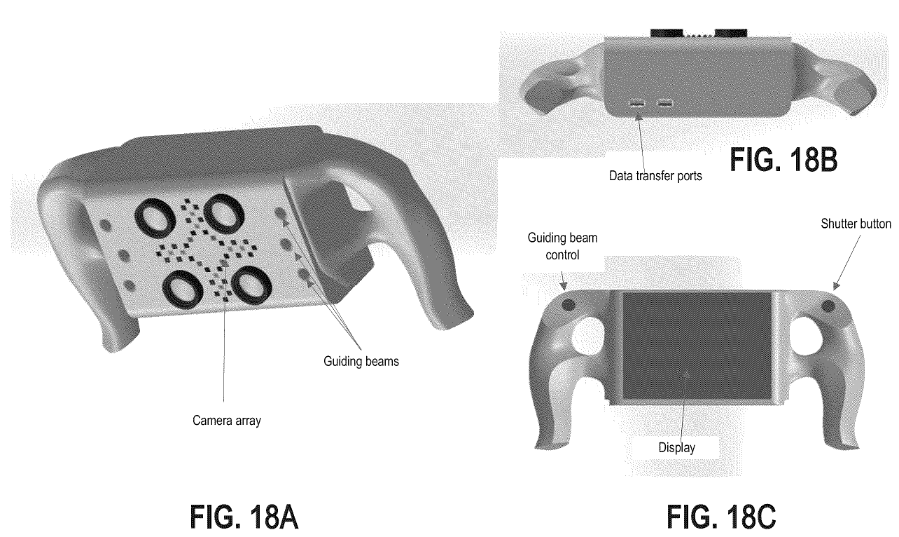

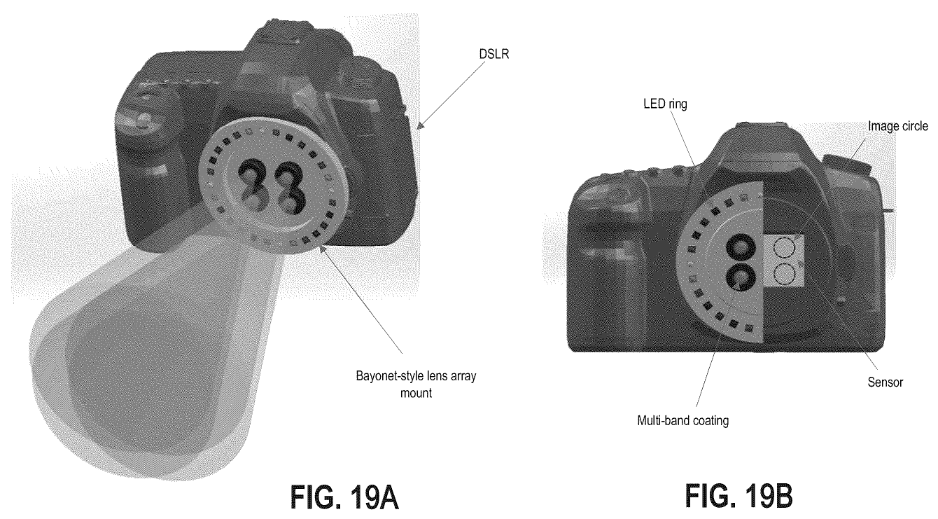

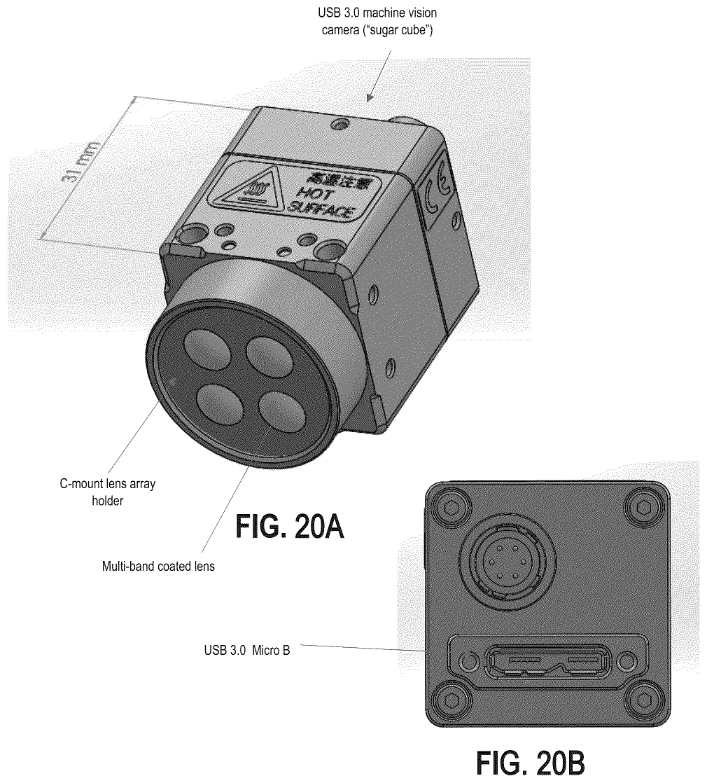

The housing 305 of the imaging system 300 may be encased in another housing. For example, handheld implementations may enclose the system within a housing optionally with one or more handles shaped to facilitate stable holding of the imaging system 300. Example handheld implementations are depicted in greater detail in FIGS. 18A-18C and in FIGS. 19A-19B. The upper surface of the housing 305 includes four openings 320A-320D. A different multi-bandpass filter 325A-325D is positioned over each opening 320A-320D and held in place by a filter cap 330A-330B. The multi-bandpass filters 325A-325D may be curved, and each pass a common waveband and at least one unique waveband, as described herein, in order to achieve high precision multi-spectral imaging across a greater number of spectral channels than would otherwise be captured by the image sensor due to its overlying color filter array. The image sensor, imaging lenses, and color filters described above are positioned within the camera housings 345A-345D. In some embodiments, a single camera housing may enclose the image sensors, imaging lenses, and color filters described above, for example, as shown in FIGS. 20A-20B. In the depicted implementation separate sensors are thus used (e.g., one sensor within each camera housing 345A-345D), but it will be appreciated that a single image sensor spanning across all of the regions exposed through the openings 320A-320D could be used in other implementations. The camera housings 345A-345D are secured to the system housing 305 using supports 340 in this embodiment, and can be secured using other suitable means in various implementations.

The upper surface of the housing 305 supports an optional illumination board 310 covered by an optical diffusing element 315. The illumination board 310 is described in further detail with respect to FIG. 4E, below. The diffusing element 315 can be composed of glass, plastic, or other optical material for diffusing light emitted from the illumination board 310 such that the object space receives substantially spatially-even illumination. Even illumination of the target object can be beneficial in certain imaging applications, for example clinical analysis of imaged tissue, because it provides, within each wavelength, a substantially even amount of illumination across the object surface. In some embodiments, the imaging systems disclosed herein may utilize ambient light instead of or in addition to light from the optional illumination board.

Due to heat generated by the illumination board 310 in use, the imaging system 300 includes a heat sink 350 including a number of heat dissipating fins 355. The heat dissipating fins 355 can extend into the space between the camera housings 345A-345D, and the upper portion of the heat sink 350 can draw heat from the illumination board 310 to the fins 355. The heat sink 350 can be made from suitable thermally conductive materials. The heat sink 350 may further help to dissipate heat from other components such that some implementations of imaging systems may be fanless.

A number of supports 365 in the housing 305 secure a processing board 335 in communication with the cameras 345A-345D. The processing board 335 can control operation of the imaging system 300. Although not illustrated, the imaging system 300 can also be configured with one or more memories, for example storing data generated by use of the imaging system and/or modules of computer-executable instructions for system control. The processing board 335 can be configured in a variety of ways, depending upon system design goals. For example, the processing board can be configured (e.g., by a module of computer-executable instructions) to control activation of particular LEDs of the illumination board 310. Some implementations can use a highly stable synchronous step-down LED driver, which can enable software control of analog LED current and also detect LED failure. Some implementations can additionally provide image data analysis functionality to the processing board (e.g., by modules of computer-executable instructions) 335 or to a separate processing board. Although not illustrated, the imaging system 300 can include data interconnects between the sensors and the processing board 335 such that the processing board 335 can receive and process data from the sensors, and between the illumination board 310 and the processing board 335 such that the processing board can drive activation of particular LEDs of the illumination board 310.

FIG. 4E depicts an example illumination board 310 that may be included in the imaging system 300, in isolation from the other components. The illumination board 310 includes four arms extending from a central region, with LEDs positioned along each arm in three columns. The spaces between LEDs in adjacent columns are laterally offset from one another to create separation between adjacent LEDs. Each column of LEDs includes a number of rows having different colors of LEDs. Four green LEDs 371 are positioned in the center region, with one green LED in each corner of the center region. Starting from the innermost row (e.g., closest to the center), each column includes a row of two deep red LEDs 372 (for a total of eight deep red LEDs). Continuing radially outward, each arm has a row of one amber LED 374 in the central column, a row of two short blue LEDs 376 in the outermost columns (for a total of eight short blue LEDs), another row of one amber LED 374 in the central column (for a total of eight amber LEDs), a row having one non-PPG NIR LED 373 and one red LED 375 in the outermost columns (for a total of four of each), and one PPG NIR LED 377 in the central column (for a total of four PPG NIR LEDs). A “PPG” LED refers to an LED activated during a number of sequential exposure for capturing photoplethysmographic (PPG) information representing pulsatile blood flow in living tissue. It will be understood that a variety of other colors and/or arrangements thereof may be used in illumination boards of other embodiments.

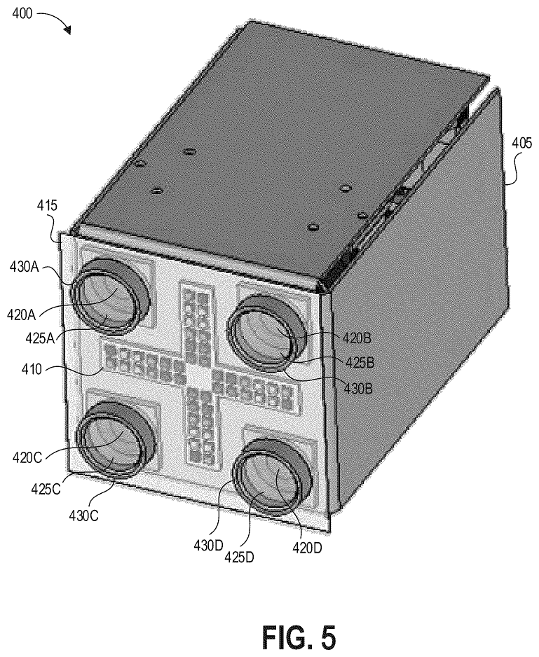

FIG. 5 depicts another embodiment of a multispectral multi-aperture imaging system, with an optical design as described with respect to FIGS. 3A and 3B. Similar to the design of the imaging system 300, the imaging system 400 includes four light paths, here shown as openings 420A-420D having multi-bandpass filter lens groups 425A-425D, which are secured to housing 405 by retaining rings 430A-430D. The imaging system 400 also includes an illumination board 410 secured to the front face of the housing 405 between the retaining rings 430A-430D, and a diffuser 415 positioned over the illumination board 410 to assist with emitting spatially even light onto the target object.

The illumination board 410 of the system 400 includes four branches of LEDs in a cross shape, with each branch including two columns of closely-spaced LEDs. Thus, the illumination board 410 is more compact than the illumination board 310 described above, and may be suitable for use with imaging systems having smaller form factor requirements. In this example configuration, each branch includes an outermost row having one green LED and one blue LED, and moving inwards includes two rows of yellow LEDs, a row of orange LEDs, a row having one red LED and one deep red LED, and a row having one amber LED and one NIR LED. Accordingly, in this implementation the LEDs are arranged such that LEDs that emit light of longer wavelengths are in the center of the illumination board 410, while LEDs that emit light of shorter wavelengths are at the edges of the illumination board 410.

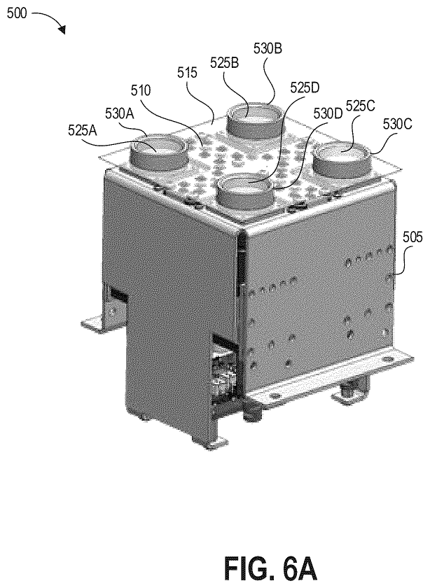

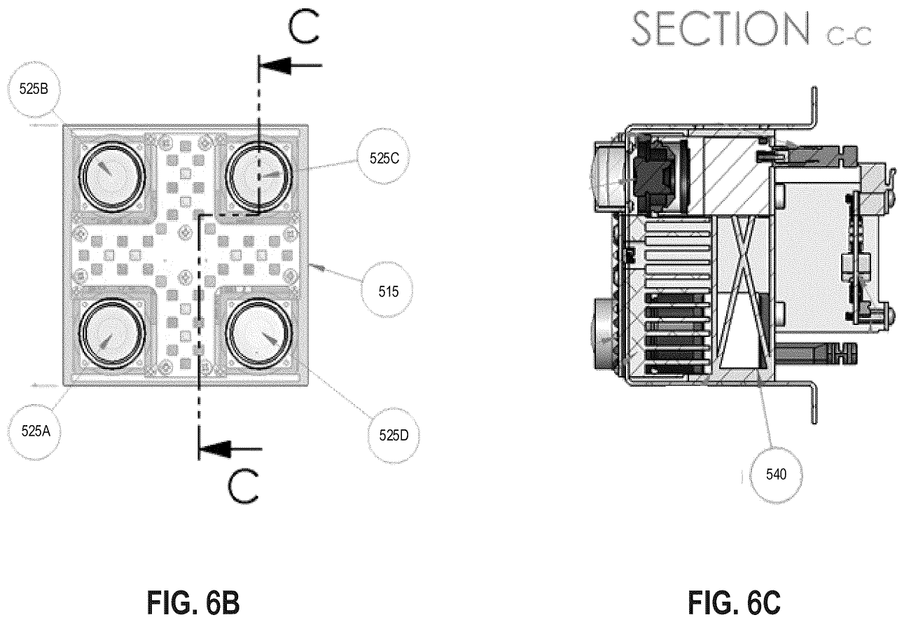

FIGS. 6A-6C depict another embodiment of a multispectral multi-aperture imaging system 500, with an optical design as described with respect to FIGS. 3A and 3B. Specifically, FIG. 6A depicts a perspective view of the imaging system 500, FIG. 6B depicts a front view of the imaging system 500, and FIG. 6C depicts a cutaway side view of the imaging system 500, cut along line C-C illustrated in FIG. 6B. The imaging system 500 includes similar components to those described above with respect to imaging system 300 (e.g., a housing 505, illumination board 510, diffusing plate 515, multi-bandpass filters 525A-525D secured over openings via retaining rings 530A-530D), but depicts a shorter form factor (e.g., in an embodiment with fewer and/or smaller embedded computing components). The system 500 also includes a direct camera-to-frame mount 540 for added rigidity and robustness of camera alignment.

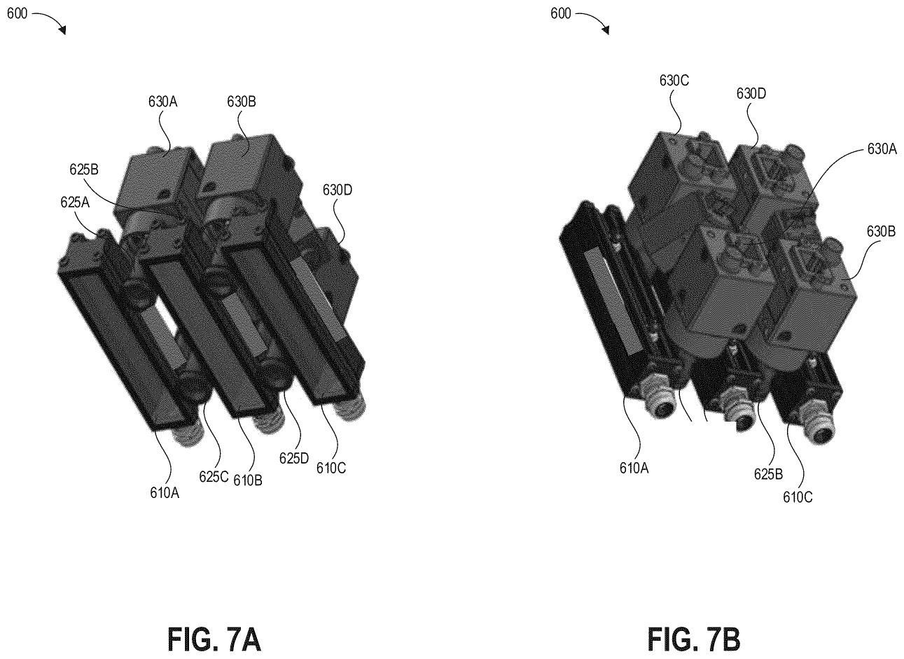

FIGS. 7A-7B depict another embodiment of a multispectral multi-aperture imaging system 600. FIGS. 7A-7B illustrate another possible arrangement of light sources 610A-610C around a multi-aperture imaging system 600. As depicted, four lens assemblies with multi-bandpass filters 625A-625D with an optical design as described with respect to FIGS. 3A-3D can be disposed in a rectangular or square configuration to provide light to four cameras 630A-630D (including image sensors). Three rectangular light emitting elements 610A-610C can be disposed parallel to one another outside of and between the lens assemblies with multi-bandpass filters 625A-625D. These can be broad-spectrum light emitting panels or arrangements of LEDs that emit discrete wavebands of light.

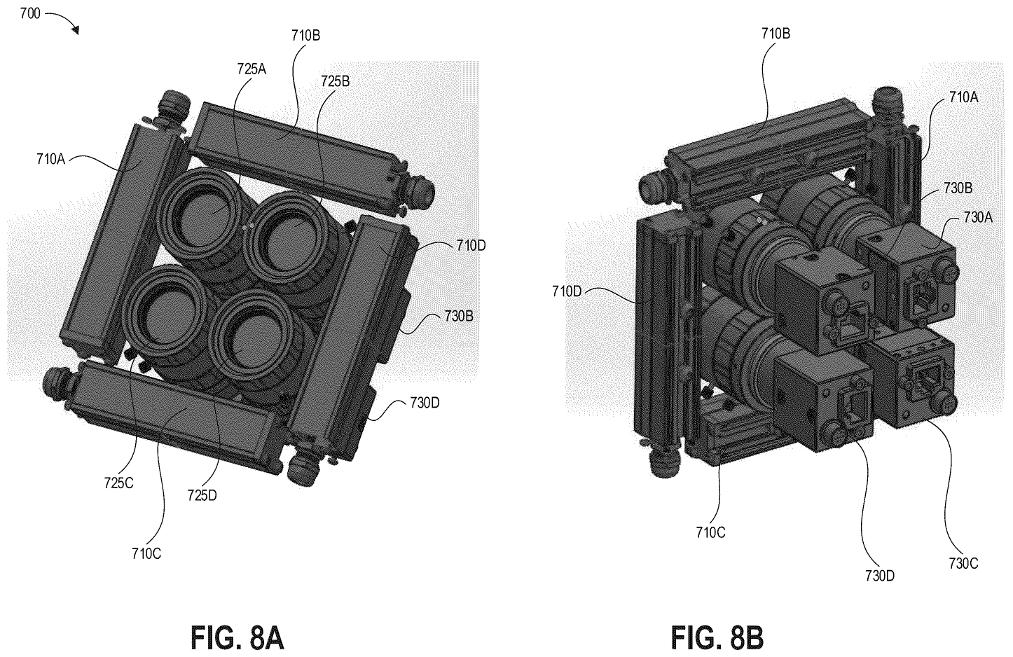

FIGS. 8A-8B depict another embodiment of a multispectral multi-aperture imaging system 700. FIGS. 8A-8B illustrate another possible arrangement of light sources 710A-710D around a multi-aperture imaging system 700. As depicted, four lens assemblies with multi-bandpass filters 725A-725D, employing an optical design as described with respect to FIGS. 3A-3D, can be disposed in a rectangular or square configuration to provide light to four cameras 730A-730D (including image sensors). The four cameras 730A-730D are illustrated in a closer example configuration which may minimize perspective differences between the lenses. Four rectangular light emitting elements 710A-710D can be positioned in a square surrounding the lens assemblies with multi-bandpass filters 725A-725D. These can be broad-spectrum light emitting panels or arrangements of LEDs that emit discrete wavebands of light.

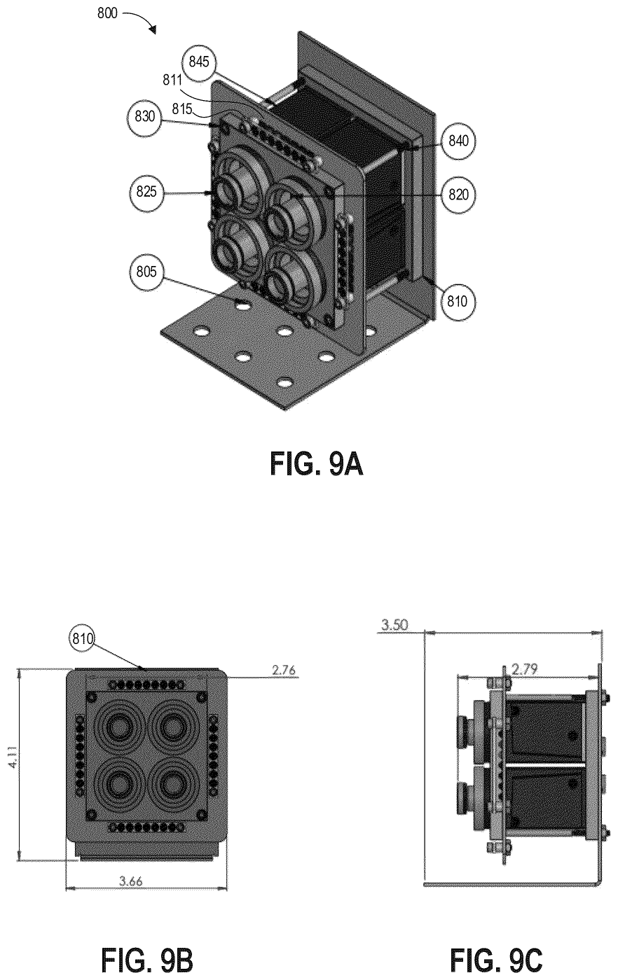

FIGS. 9A-9C depict another embodiment of a multispectral multi-aperture imaging system 800. The imaging system 800 includes a frame 805 coupled to a lens cluster frame front 830 that includes openings 820 and support structures for micro-video lenses 825, which can be provided with multi-bandpass filters using an optical design as described with respect to FIGS. 3A-3D. The micro-video lenses 825 provide light to four cameras 845 (including imaging lenses and image sensor regions) mounted on a lens cluster frame back 840. Four linear arrangements of LEDs 811 are disposed along the four sides of the lens cluster frame front 830, each provided with its own diffusing element 815. FIGS. 9B and 9C depict example dimensions in inches to show one possible size of the multi-aperture imaging system 800.

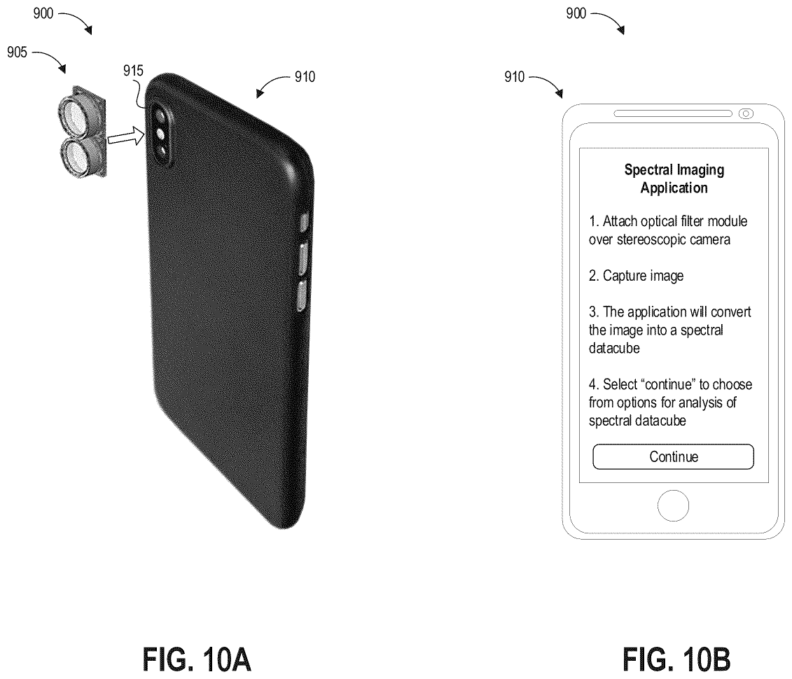

FIG. 10A depicts another embodiment of a multispectral multi-aperture imaging system 900, with an optical design as described with respect to FIGS. 3A-3D. The imaging system 900 can be implemented as a set of multi-bandpass filters 905 that are attachable over a multi-aperture camera 915 of a mobile device 910. For example, certain mobile devices 910 such as smartphones can be equipped with stereoscopic imaging systems having two openings leading to two image sensor regions. The disclosed multi-aperture spectral imaging techniques can be implemented in such devices by providing them with a suitable set of multi-bandpass filters 905 to pass multiple narrower wavebands of light to the sensor regions. Optionally, the set of multi-bandpass filters 905 can be equipped with an illuminant (such as an LED array and diffuser) that provides light at these wavebands to the object space.

The system 900 can also include a mobile application that configures the mobile device to perform the processing that generates the multispectral datacube, as well as processing the multispectral datacube (e.g., for clinical tissue classification, biometric recognition, materials analysis, or other applications). Alternatively, the mobile application may configure the device 910 to send the multispectral datacube over a network to a remote processing system, and then receive and display a result of the analysis. An example user interface 910 for such an application is shown in FIG. 10B.

FIGS. 11A-11B depict an example set of wavebands that can be passed by the filters of four-filter implementations of the multispectral multi-aperture imaging systems of FIGS. 3A-10B, for example to an image sensor having the Bayer CFA (or another RGB or RGB-IR CFA). The spectral transmission response of wavebands as passed by the multi-bandpass filters are shown by the solid lines in the graphs 1000 of FIG. 11A and are denoted by Tₙ^{λ}, where n represents the camera number, ranging from 1 through 4. The dashed lines represent the combined spectral response of Tₙ^{λ} with either the spectral transmission of a green pixel, Q_{G}^{λ}, a red pixel, Q_{R}^{λ}, or a blue pixel, Q_{B}^{λ}, that would be present in a typical Bayer CFA. These transmission curves also include the effects of quantum efficiency due to the sensor used in this example. As illustrated, this set of four cameras collectively captures eight unique channels or wavebands. Each filter passes two common wavebands (the two left-most peaks) to the respective cameras, as well as two additional wavebands. In this implementation, the first and third cameras receive light in a first shared NIR waveband (the right-most peak), and the second and fourth cameras receive light in a second shared NIR waveband (the peak second-most to the right). Each of the cameras also receives one unique waveband ranging from approximately 550 nm or 550 nm to approximately 800 nm or 800 nm. Thus, the camera can capture eight unique spectral channels using a compact configuration. A graph 1010 in FIG. 11B depicts the spectral irradiance of an LED board as described in FIG. 4E that may be used as illumination for the 4 cameras shown in FIG. 11A.

In this implementation, the eight wavebands have been selected based on producing spectral channels suitable for clinical tissue classification, and may also be optimized with respect to signal-to-noise ratio (SNR) and frame rate while limiting the number of LEDS (which introduce heat into the imaging system). The eight wavebands include a common waveband of blue light (the leftmost peak in the graphs 1000) that is passed by all four filters, because tissue (e.g., animal tissue including human tissue) exhibits higher contrast at blue wavelengths than at green or red wavelengths. Specifically, human tissue exhibits its highest contrast when imaged at a waveband centered on around 420 nm, as shown in the graphs 1000. Because the channel corresponding to the common waveband is used for disparity correction, this higher contrast can produce more accurate correction. For example in disparity correction the image processor can employ local or global methods to find a set of disparities so that a figure of merit corresponding to similarity between local image patches or images is maximized. Alternatively, the image processor can employ similar methods that minimize a figure of merit corresponding to dissimilarity. These figures of merit can be based on entropy, correlation, absolute differences, or on deep learning methods. Global methods of disparity calculation can operate iteratively, terminating when the figure of merit is stable. Local methods can be used to calculate disparity point by point, using a fixed patch in one image as an input into the figure of merit and a number of different patches, each determined by a different value of disparity under test, from the other image. All such methods can have constraints imposed on the range of disparities that are considered. These constraints can be based on knowledge of the object depth and distance, for instance. The constraints could also be imposed based on a range of gradients expected in an object. Constraints on the calculated disparities can also be imposed by projective geometry, such as the epipolar constraint. Disparity can be calculated at multiple resolutions, with the output of disparities calculated at lower resolutions acting as initial values or constraints on the disparities calculated at the next level of resolution. For instance, a disparity calculated at a resolution level of 4 pixels in one calculation can be used to set constraints of +4 pixels in a next calculation of disparity at higher resolution. All algorithms that calculate from disparity will benefit from higher contrast, particularly if that source of contrast is correlated for all viewpoints. Generally speaking, the common waveband can be selected based on corresponding to the highest contrast imaging of the material that is expected to be imaged for a particular application.

After image capture, color separation between adjacent channels may not be perfect, and so this implementation also has an additional common waveband passed by all filters—depicted in the graphs 1000 as the green waveband adjacent to the blue waveband. This is because blue color filter pixels are sensitive to retentions of the green spectrum due to its broad spectral bandpass. This typically manifests as spectral overlap, which may also be characterized as intentional crosstalk, between adjacent RGB pixels. This overlap enables the spectral sensitivity of color cameras to be similar to the spectral sensitivity of a human retina, such that the resultant color space is qualitatively similar to human vision. Accordingly, having a common green channel can enable separation of the portion of the signal generated by blue photodiodes that truly corresponds to received blue light, by separating out the portion of the signal due to green light. This can be accomplished using spectral unmixing algorithms that factor in the transmittance (shown in the legend by T with a solid black line) of the multi-band pass filter, the transmittance of the corresponding CFA color filter (shown in the legend by Q with dashed red, green, and blue lines). It will be appreciated that some implementations may use red light as a common waveband, and in such instances a second common channel may not be necessary.

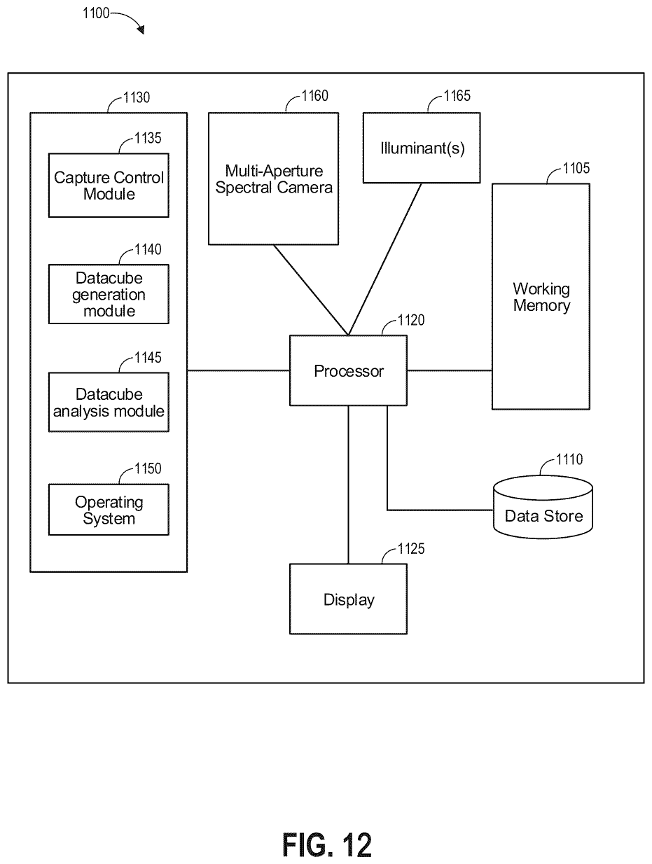

FIG. 12 illustrates a high-level block diagram of an example compact imaging system 1100 with high resolution spectral imaging capabilities, the system 1100 having a set of components including a processor 1120 linked to an multi-aperture spectral camera 1160 and illuminant(s) 1165. A working memory 1105, storage 1110, electronic display 1125, and memory 1130 are also in communication with the processor 1120. As described herein, the system 1100 may capture a greater number of image channels than there are different colors of filters in the CFA of the image sensor by using different multi-bandpass filters placed over different openings of the multi-aperture spectral camera 1160.

System 1100 may be a device such as cell phone, digital camera, tablet computer, personal digital assistant, or the like. System 1100 may also be a more stationary device such as a desktop personal computer, video conferencing station, or the like that uses an internal or external camera for capturing images. System 1100 can also be a combination of an image capture device and a separate processing device receiving image data from the image capture device. A plurality of applications may be available to the user on system 1100. These applications may include traditional photographic applications, capture of still images and video, dynamic color correction applications, and brightness shading correction applications, among others.

The image capture system 1100 includes the multi-aperture spectral camera 1160 for capturing images. The multi-aperture spectral camera 1160 can be, for example, any of the devices of FIGS. 3A-10B. The multi-aperture spectral camera 1160 may be coupled to the processor 1120 to transmit captured images in different spectral channels and from different sensor regions to the image processor 1120. The illuminant(s) 1165 can also be controlled by the processor to emit light at certain wavelengths during certain exposures, as described in more detail below. The image processor 1120 may be configured to perform various operations on a received captured image in order to output a high quality, disparity corrected multispectral datacube.

Processor 1120 may be a general purpose processing unit or a processor specially designed for imaging applications. As shown, the processor 1120 is connected to a memory 1130 and a working memory 1105. In the illustrated embodiment, the memory 1130 stores a capture control module 1135, datacube generation module 1140, datacube analysis module 1145, and operating system 1150. These modules include instructions that configure the processor to perform various image processing and device management tasks. Working memory 1105 may be used by processor 1120 to store a working set of processor instructions contained in the modules of memory 1130. Alternatively, working memory 1105 may also be used by processor 1120 to store dynamic data created during the operation of device 1100.

As mentioned above, the processor 1120 is configured by several modules stored in the memory 1130. The capture control module 1135 includes instructions that configure the processor 1120 to adjust the focus position of the multi-aperture spectral camera 1160, in some implementations. The capture control module 1135 also includes instructions that configure the processor 1120 to capture images with the multi-aperture spectral camera 1160, for example multispectral images captured at different spectral channels as well as PPG images captured at the same spectral channel (e.g., a NIR channel). Non-contact PPG imaging normally uses near-infrared (NIR) wavelengths as illumination to take advantage of the increased photon penetration into the tissue at this wavelength. Therefore, processor 1120, along with capture control module 1135, multi-aperture spectral camera 1160, and working memory 1105 represent one means for capturing a set of spectral images and/or a sequence of images.

The datacube generation module 1140 includes instructions that configure the processor 1120 to generate a multispectral datacube based on intensity signals received from the photodiodes of different sensor regions. For example, the datacube generation module 1140 can estimate a disparity between the same regions of an imaged object based on a spectral channel corresponding to the common waveband passed by all multi-bandpass filters, and can use this disparity to register all spectral images across all captured channels to one another (e.g., such that the same point on the object is represented by substantially the same (x,y) pixel location across all spectral channels). The registered images collectively form the multispectral datacube, and the disparity information may be used to determine depths of different imaged objects, for example a depth difference between healthy tissue and a deepest location within a wound site. In some embodiments, the datacube generation module 1140 may also perform spectral unmixing to identify which portions of the photodiode intensity signals correspond to which passed wavebands, for example based on spectral unmixing algorithms that factor in filter transmittances and sensor quantum efficiency.

The datacube analysis module 1145 can implement various techniques to analyze the multispectral datacube generated by the datacube generation module 1140, depending upon the application. For example, some implementations of the datacube analysis module 1145 can provide the multispectral datacube (and optionally depth information) to a machine learning model trained to classify each pixel according to a certain state. These states may be clinical states in the case of tissue imaging, for example burn states (e.g., first degree burn, second degree burn, third degree burn, or healthy tissue categories), wound states (e.g., hemostasis, inflammation, proliferation, remodeling or healthy skin categories), healing potential (e.g., a score reflecting the likelihood that the tissue will heal from a wounded state, with or without a particular therapy), perfusion states, cancerous states, or other wound-related tissue states. The datacube analysis module 1145 can also analyze the multispectral datacube for biometric recognition and/or materials analysis.

Operating system module 1150 configures the processor 1120 to manage the memory and processing resources of the system 1100. For example, operating system module 1150 may include device drivers to manage hardware resources such as the electronic display 1125, storage 1110, multi-aperture spectral camera 1160, or illuminant(s) 1165. Therefore, in some embodiments, instructions contained in the image processing modules discussed above may not interact with these hardware resources directly, but instead interact through standard subroutines or APIs located in operating system component 1150. Instructions within operating system 1150 may then interact directly with these hardware components.

The processor 1120 may be further configured to control the display 1125 to display the captured images and/or a result of analyzing the multispectral datacube (e.g., a classified image) to a user. The display 1125 may be external to an imaging device including the multi-aperture spectral camera 1160 or may be part of the imaging device. The display 1125 may also be configured to provide a view finder for a user prior to capturing an image. The display 1125 may comprise an LCD or LED screen, and may implement touch sensitive technologies.

Processor 1120 may write data to storage module 1110, for example data representing captured images, multispectral datacubes, and datacube analysis results. While storage module 1110 is represented graphically as a traditional disk device, those with skill in the art would understand that the storage module 1110 may be configured as any storage media device. For example, the storage module 1110 may include a disk drive, such as a floppy disk drive, hard disk drive, optical disk drive or magneto-optical disk drive, or a solid state memory such as a FLASH memory, RAM, ROM, and/or EEPROM. The storage module 1110 can also include multiple memory units, and any one of the memory units may be configured to be within the image capture device 1100, or may be external to the image capture system 1100. For example, the storage module 1110 may include a ROM memory containing system program instructions stored within the image capture system 1100. The storage module 1110 may also include memory cards or high speed memories configured to store captured images which may be removable from the camera.

Although FIG. 12 depicts a system comprising separate components to include a processor, imaging sensor, and memory, one skilled in the art would recognize that these separate components may be combined in a variety of ways to achieve particular design objectives. For example, in an alternative embodiment, the memory components may be combined with processor components to save cost and improve performance.

Additionally, although FIG. 12 illustrates two memory components-memory component 1130 comprising several modules and a separate memory 1105 comprising a working memory-one with skill in the art would recognize several embodiments utilizing different memory architectures. For example, a design may utilize ROM or static RAM memory for the storage of processor instructions implementing the modules contained in memory 1130. Alternatively, processor instructions may be read at system startup from a disk storage device that is integrated into system 1100 or connected via an external device port. The processor instructions may then be loaded into RAM to facilitate execution by the processor. For example, working memory 1105 may be a RAM memory, with instructions loaded into working memory 1105 before execution by the processor 1120.

### Overview of Example Image Processing Techniques

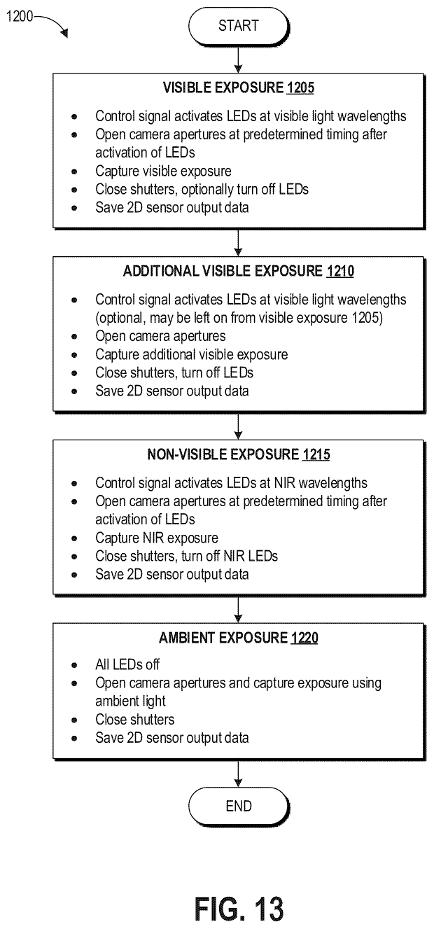

FIG. 13 is a flowchart of an example process 1200 for capturing image data using the multispectral multi-aperture imaging systems of FIGS. 3A-10B and 12. FIG. 13 depicts four example exposures that can be used to generate a multispectral datacube as described herein-a visible exposure 1205, an additional visible exposure 1210, a non-visible exposure 1215, and an ambient exposure 1220. It will be appreciated that these may be captured in any order, and some exposures may be optionally removed from or added to a particular workflow as described below. Further, the process 1200 is described with reference to the wavebands of FIGS. 11A and 11B, however similar workflows can be implemented using image data generated based on other sets of wavebands. Additionally, flat field correction may further be implemented in accordance with various known flat field correction techniques, to improve image acquisition and/or disparity correction in various embodiments.

For the visible exposure 1205, LEDs of first five peaks (the left five peaks corresponding to visible light in the graphs 1000 of FIG. 11A) can be turned on by a control signal to the illumination board. The wave of light output may need to stabilize, at a time specific to particular LEDs, for example 10 ms. The capture control module 1135 can begin the exposure of the four cameras after this time and can continue this exposure for a duration of around 30 ms, for example. Thereafter, the capture control module 1135 can cease the exposure and pull the data off of the sensor regions (e.g., by transferring raw photodiode intensity signals to the working memory 1105 and/or data store 1110). This data can include a common spectral channel for use in disparity correction as described herein.

In order to increase the SNR, some implementations can capture the additional visible exposure 1210 using the same process described for the visible exposure 1205. Having two identical or near-identical exposures can increase the SNR to yield more accurate analysis of the image data. However, this may be omitted in implementations where the SNR of a single image is acceptable. A duplicate exposure with the common spectral channel may also enable more accurate disparity correction in some implementations.

Some implementations can also capture a non-visible exposure 1215 corresponding to NIR or IR light. For example, the capture control module 1135 can activate two different NIR LEDs corresponding to the two NIR channels shown in FIG. 11A. The wave of light output may need to stabilize, at a time specific to particular LEDs, for example 10 ms. The capture control module 1135 can begin the exposure of the four cameras after this time and continue this exposure for a duration of around 30 ms, for example. Thereafter, the capture control module 1135 can cease the exposure and pull the data off of the sensor regions (e.g., by transferring raw photodiode intensity signals to the working memory 1105 and/or data store 1110). In this exposure, there may be no common waveband passed to all sensor regions, as it can safely be assumed that there is no change in the shape or positioning of the object relative to the exposures 1205, 1210 and, thus previously computed disparity values can be used to register the NIR channels.

In some implementations, multiple exposures can be captured sequentially to generate PPG data representing the change in shape of a tissue site due to pulsatile blood flow. These PPG exposures may be captured at a non-visible wavelength in some implementations. Although the combination of PPG data with multispectral data may increase the accuracy of certain medical imaging analyses, the capture of PPG data can also introduce additional time into the image capture process. This additional time can introduce errors due to movement of the handheld imager and/or object, in some implementations. Thus, certain implementations may omit capture of PPG data.

Some implementations can additionally capture the ambient exposure 1220. For this exposure, all LEDs can be turned off to capture an image using ambient illumination (e.g., sunlight, light from other illuminant sources). The capture control module 1135 can begin the exposure of the four cameras after this time and can keep the exposure ongoing for a desired duration of, for example, around 30 ms. Thereafter, the capture control module 1135 can cease the exposure and pull the data off of the sensor regions (e.g., by transferring raw photodiode intensity signals to the working memory 1105 and/or data store 1110). The intensity values of the ambient exposure 1220 can be subtracted from the values of the visible exposure 1205 (or the visible exposure 1205 corrected for SNR by the second exposure 1210) and also from the non-visible exposure 1215 in order to remove the influence of ambient light from the multispectral datacube. This can increase the accuracy of downstream analysis by isolating the portion of the generated signals that represent light emitted by the illuminants and reflected from the object/tissue site. Some implementations may omit this step if analytical accuracy is sufficient using just the visible 1205, 1210 and non-visible 1215 exposures.

It will be appreciated that the particular exposure times listed above are examples of one implementation, and that in other implementations exposure time can vary depending upon the image sensor, illuminant intensity, and imaged object.

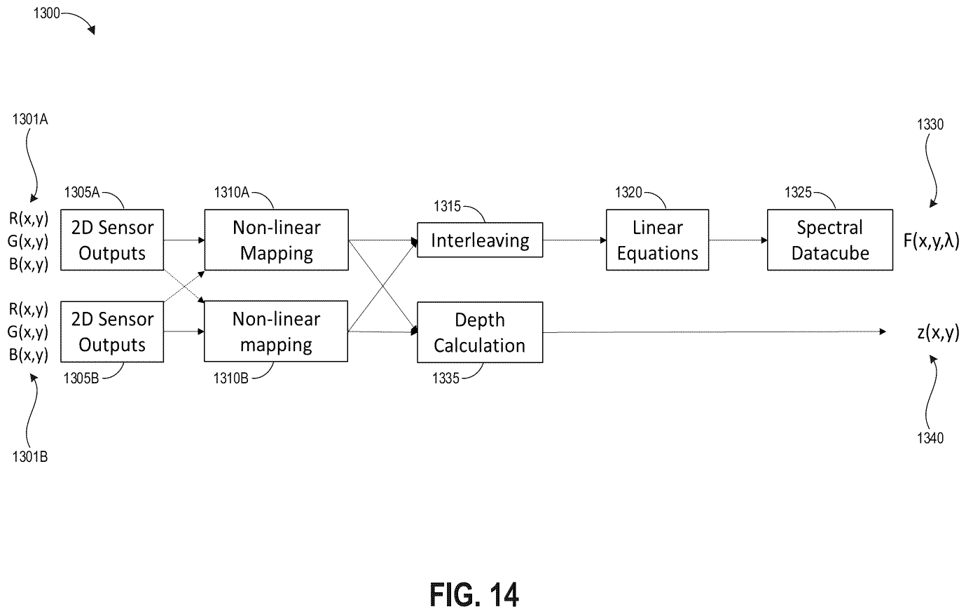

FIG. 14 depicts a schematic block diagram of a workflow 1300 for processing image data, for example image data captured using the process 1200 of FIG. 13 and/or using the multispectral multi-aperture imaging systems of FIGS. 3A-10B and 12. The workflow 1300 shows the output of two RGB sensor regions 1301A, 1301B, however the workflow 1300 can be extended to greater numbers of sensor regions and sensor regions corresponding to different CFA color channels.

The RGB sensor outputs from the two sensor regions 1301A, 1301B are stored at the 2D sensor outputs modules 1305A, 1305B, respectively. The values of both sensor regions are sent to the non-linear mapping modules 1310A, 1310B, which can perform disparity correction by identifying disparity between the captured images using the common channel and then applying this determined disparity across all channels to register all spectral images to one another.

The outputs of both non-linear mapping modules 1310A, 1310B are then provided to the depth calculation module 1335, which can compute a depth of a particular region of interest in the image data. For example, the depth may represent the distance between the object and the image sensor. In some implementations, multiple depth values can be computed and compared to determine the depth of the object relative to something other than the image sensor. For example, a greatest depth of a wound bed can be determined, as well as a depth (greatest, lowest, or average) of healthy tissue surrounding the wound bed. By subtracting the depth of the healthy tissue from the depth of the wound bed, the deepest depth of the wound can be determined. This depth comparison can additionally be performed at other points in the wound bed (e.g., all or some predetermined sampling) in order to build a 3D map of the depth of the wound at various points (shown in FIG. 14 as z (x,y) where z would be a depth value). In some embodiments, greater disparity may improve the depth calculation, although greater disparity may also result in more computationally intensive algorithms for such depth calculations.

The outputs of both non-linear mapping modules 1310A, 1310B are also provided to the linear equations module 1320, which can treat the sensed values as set of linear equations for spectral unmixing. One implementation can use the Moore-Penrose pseudo-inverse equation as a function of at least sensor quantum efficiency and filter transmittance values to compute actual spectral values (e.g., intensity of light at particular wavelengths that were incident at each (x,y) image point). This can be used in implementations that require high accuracy, such as clinical diagnostics and other biological applications. Application of the spectral unmixing can also provide an estimate of photon flux and SNR.

Based on the disparity-corrected spectral channel images and the spectral unmixing, the workflow 1300 can generate a spectral datacube 1325, for example in the illustrated format of F(x,y,λ) where F represents the intensity of light at a specific (x,y) image location at a specific wavelength or waveband λ.

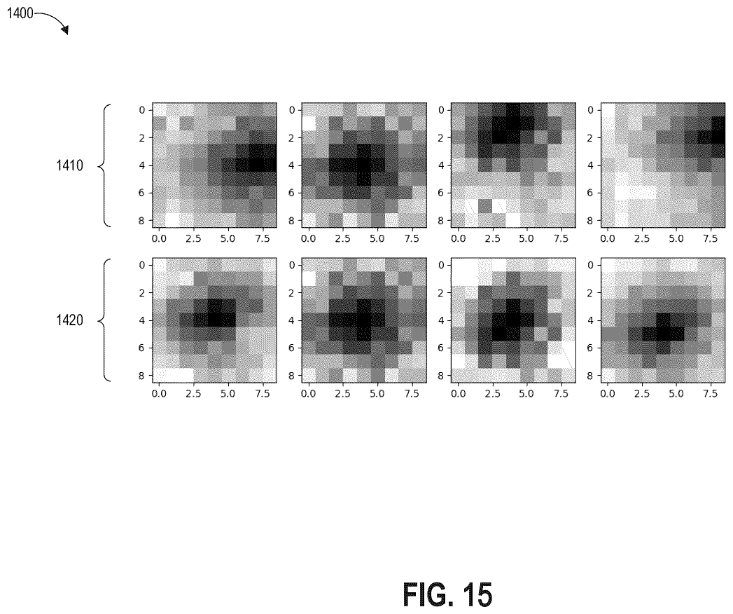

FIG. 15 graphically depicts disparity and disparity correction for processing image data, for example image data captured using the process of FIG. 13 and/or using the multispectral multi-aperture imaging systems of FIGS. 3A-10B and 12. The first set of images 1410 show image data of the same physical location on an object as captured by four different sensor regions. As illustrated, this object location is not in the same location across the raw images, based on the (x,y) coordinate frames of the photodiode grids of the image sensor regions. The second set of images 1420 shows that same object location after disparity correction, which is now in the same (x,y) location in the coordinate frame of the registered images. It will be appreciated that such registration may involve cropping certain data from edge regions of the images that do not entirely overlap with one another.

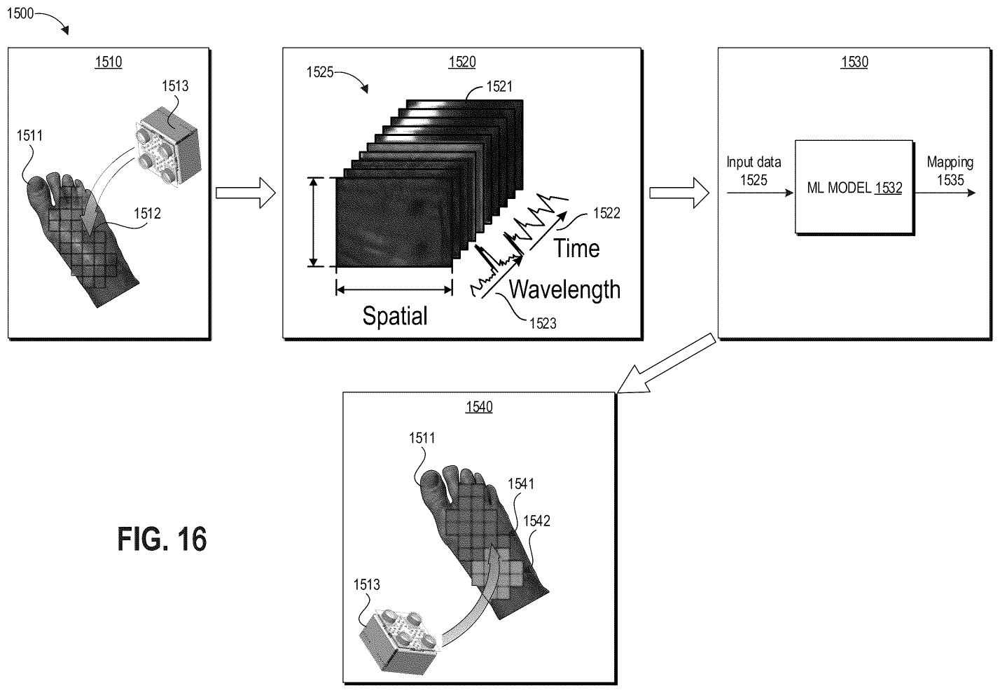

FIG. 16 graphically depicts a workflow 1500 for performing pixel-wise classification on multispectral image data, for example image data captured using the process of FIG. 13, processed according to FIGS. 14 and 15, and/or using the multispectral multi-aperture imaging systems of FIGS. 3A-10B and 12.

At block 1510, the multispectral multi-aperture imaging system 1513 can capture image data representing physical points 1512 on an object 1511. In this example, the object 1511 includes tissue of a patient that has a wound. A wound can comprise a burn, a diabetic ulcer (e.g., a diabetic foot ulcer), a non-diabetic ulcer (e.g., pressure ulcers or slow-healing wounds), a chronic ulcer, a post-surgical incision, an amputation site (before or after the amputation procedure), a cancerous lesion, or damaged tissue. Where PPG information is included, the disclosed imaging systems provide a method to assess pathologies involving changes to tissue blood flow and pulse rate including: tissue perfusion; cardiovascular health; wounds such as ulcers; peripheral arterial disease, and respiratory health.

At block 1520, the data captured by the multispectral multi-aperture imaging system 1513 can be processed into a multispectral datacube 1525 having a number of different wavelengths 1523, and, optionally, a number of different images at the same wavelength corresponding to different times (PPG data 1522). For example, the image processor 1120 can be configured by the datacube generation module 1140 to generate the multispectral datacube 1525 according to the workflow 1300. Some implementations may also associated depth values with various points along the spatial dimensions, as described above.

At block 1530, the multispectral datacube 1525 can be analyzed as input data 1525 into a machine learning model 1532 to generate a classified mapping 1535 of the imaged tissue. The classified mapping can assign each pixel in the image data (which, after registration, represent specific points on the imaged object 1511) to a certain tissue classification, or to a certain healing potential score. The different classifications and scores can be represented using visually distinct colors or patterns in the output classified image. Thus, even though a number of images are captured of the object 1511, the output can be a single image of the object (e.g., a typical RGB image) overlaid with visual representations of pixel-wise classification.

The machine learning model 1532 can be an artificial neural network in some implementations. Artificial neural networks are artificial in the sense that they are computational entities, inspired by biological neural networks but modified for implementation by computing devices. Artificial neural networks are used to model complex relationships between inputs and outputs or to find patterns in data, where the dependency between the inputs and the outputs cannot be easily ascertained. A neural network typically includes an input layer, one or more intermediate (“hidden”) layers, and an output layer, with each layer including a number of nodes. The number of nodes can vary between layers. A neural network is considered “deep” when it includes two or more hidden layers. The nodes in each layer connect to some or all nodes in the subsequent layer and the weights of these connections are typically learnt from data during the training process, for example through backpropagation in which the network parameters are tuned to produce expected outputs given corresponding inputs in labeled training data. Thus, an artificial neural network is an adaptive system that is configured to change its structure (e.g., the connection configuration and/or weights) based on information that flows through the network during training, and the weights of the hidden layers can be considered as an encoding of meaningful patterns in the data.

A fully connected neural network is one in which each node in the input layer is connected to each node in the subsequent layer (the first hidden layer), each node in that first hidden layer is connected in turn to each node in the subsequent hidden layer, and so on until each node in the final hidden layer is connected to each node in the output layer.

A CNN is a type of artificial neural network, and like the artificial neural network described above, a CNN is made up of nodes and has learnable weights. However, the layers of a CNN can have nodes arranged in three dimensions: width, height, and depth, corresponding to the 2×2 array of pixel values in each video frame (e.g., the width and height) and to the number of video frames in the sequence (e.g., the depth). The nodes of a layer may only be locally connected to a small region of the width and height layer before it, called a receptive field. The hidden layer weights can take the form of a convolutional filter applied to the receptive field. In some embodiments, the convolutional filters can be two-dimensional, and thus, convolutions with the same filter can be repeated for each frame (or convolved transformation of an image) in the input volume or for designated subset of the frames. In other embodiments, the convolutional filters can be three-dimensional and thus extend through the full depth of nodes of the input volume. The nodes in each convolutional layer of a CNN can share weights such that the convolutional filter of a given layer is replicated across the entire width and height of the input volume (e.g., across an entire frame), reducing the overall number of trainable weights and increasing applicability of the CNN to data sets outside of the training data. Values of a layer may be pooled to reduce the number of computations in a subsequent layer (e.g., values representing certain pixels may be passed forward while others are discarded), and further along the depth of the CNN pool masks may reintroduce any discarded values to return the number of data points to the previous size. A number of layers, optionally with some being fully connected, can be stacked to form the CNN architecture.

During training, an artificial neural network can be exposed to pairs in its training data and can modify its parameters to be able to predict the output of a pair when provided with the input. For example, the training data can include multispectral datacubes (the input) and classified mappings (the expected output) that have been labeled, for example by a clinician who has designated areas of the wound that correspond to certain clinical states, and/or with healing (1) or non-healing (0) labels sometime after initial imaging of the wound when actual healing is known. Other implementations of the machine learning model 1532 can be trained to make other types of predictions, for example the likelihood of a wound healing to a particular percentage area reduction over a specified time period (e.g., at least 50% area reduction within 30 days) or wound states such as, hemostasis, inflammation, proliferation, remodeling or healthy skin categories. Some implementations may also incorporate patient metrics into the input data to further increase classification accuracy, or may segment training data based on patient metrics to train different instances of the machine learning model 1532 for use with other patients having those same patient metrics. Patient metrics can include textual information or medical history or aspects thereof describing characteristics of the patient or the patient's health status, for example the area of a wound, lesion, or ulcer, the BMI of the patient, the diabetic status of the patient, the existence of peripheral vascular disease or chronic inflammation in the patient, the number of other wounds the patient has or has had, whether the patient is or has recently taken immunosuppressant drugs (e.g., chemotherapy) or other drugs that positively or adversely affect wound healing rate, HbAlc, chronic kidney failure stage IV, type II vs type I diabetes, chronic anemia, asthma, drug use, smoking status, diabetic neuropathy, deep vein thrombosis, previous myocardial infarction, transient ischemic attacks, or sleep apnea or any combination thereof. These metrics can be converted into a vector representation through appropriate processing, for example through word-to-vec embeddings, a vector having binary values representing whether the patient does or does not have the patient metric (e.g., does or does not have type I diabetes), or numerical values representing a degree to which the patient has each patient metric.

At block 1540, the classified mapping 1535 can be output to a user. In this example, the classified mapping 1535 uses a first color 1541 to denote pixels classified according to a first state and uses a second color 1542 to denote pixels classified according to a second state. The classification and resulting classified mapping 1535 may exclude background pixels, for example based on object recognition, background color identification, and/or depth values. As illustrated, some implementations of the multispectral multi-aperture imaging system 1513 can project the classified mapping 1535 back on to the tissue site. This can be particularly beneficial when the classified mapping includes a visual representation of a recommended margin and/or depth of excision.

These methods and systems may provide assistance to clinicians and surgeons in the process of dermal wound management, such as burn excision, amputation level, lesion removal, and wound triage decisions. Alternatives described herein can be used to identify and/or classify the severity of decubitus ulcers, hyperaemia, limb deterioration, Raynaud's Phenomenon, scleroderma, chronic wounds, abrasions, lacerations, hemorrhaging, rupture injuries, punctures, penetrating wounds, skin cancers, such as basal cell carcinoma, squamous cell carcinoma, melanoma, actinic keratosis, or any type of tissue change, wherein the nature and quality of the tissue differs from a normal state. The devices described herein may also be used to monitor healthy tissue, facilitate and improve wound treatment procedures, for example allowing for a faster and more refined approach for determining the margin for debridement, and evaluate the progress of recovery from a wound or disease, especially after a treatment has been applied. In some alternatives described herein, devices are provided that allow for the identification of healthy tissue adjacent to wounded tissue, the determination of an excision margin and/or depth, the monitoring of the recovery process after implantation of a prosthetic, such as a left ventricular assist device, the evaluation of the viability of a tissue graft or regenerative cell implant, or the monitoring of surgical recovery, especially after reconstructive procedures. Moreover, alternatives described herein may be used to evaluate the change in a wound or the generation of healthy tissue after a wound, in particular, after introduction of a therapeutic agent, such as a steroid, hepatocyte growth factor, fibroblast growth factor, an antibiotic, or regenerative cells, such as an isolated or concentrated cell population that comprises stem cells, endothelial cells and/or endothelial precursor cells.

### Overview of Example Distributed Computing Environment

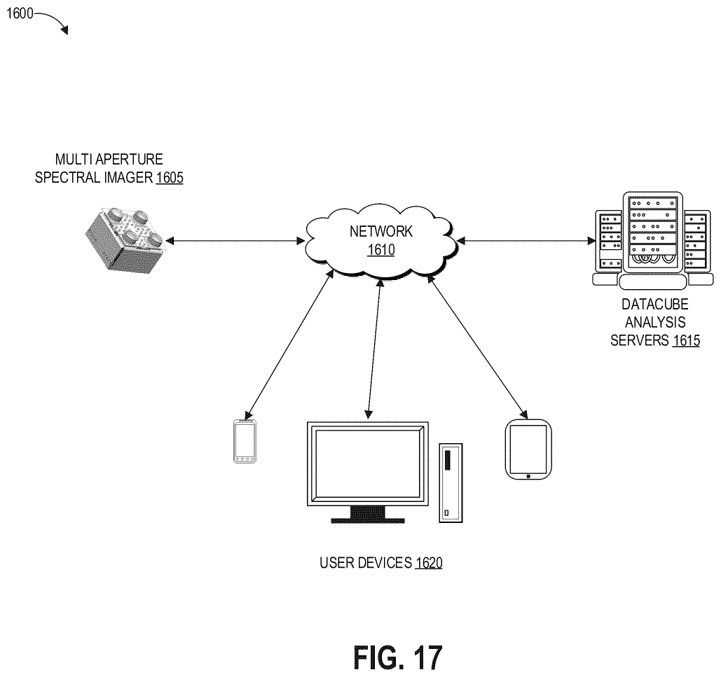

FIG. 17 depicts a schematic block diagram of an example distributed computing system 1600 including a multispectral multi-aperture imaging system 1605, which can be any of the multispectral multi-aperture imaging systems of FIGS. 3A-10B and 12. As depicted the datacube analysis servers 1615 may include one or more computers, perhaps arranged in a cluster of servers or as a server farm. The memory and processors that make up these computers may be located within one computer or distributed throughout many computers (including computers that are remote from one another).

The multispectral multi-aperture imaging system 1605 can include networking hardware (e.g., a wireless Internet, satellite, Bluetooth, or other transceiver) for communicating over the network 1610 with user devices 1620 and datacube analysis servers 1615. For example, in some implementations the processor of the multispectral multi-aperture imaging system 1605 may be configured to control image capture, and then send raw data to the datacube analysis servers 1615. Other implementations of the processor of the multispectral multi-aperture imaging system 1605 may be configured to control image capture and perform spectral unmixing and disparity correction to generate a multispectral datacube, which is then sent to the datacube analysis servers 1615. Some implementations can perform full processing and analysis locally on the multispectral multi-aperture imaging system 1605, and may send the multispectral datacube and resulting analysis to the datacube analysis servers 1615 for aggregate analysis and/or use in training or retraining machine learning models. As such, the datacube analysis servers 1615 may provide updated machine learning models to the multispectral multi-aperture imaging system 1605. The processing load of generating the end result of analyzing the multispectral datacube can be split between the multi-aperture imaging system 1605 and the datacube analysis servers 1615 in various ways, depending upon the processing power of the multi-aperture imaging system 1605.

The network 1610 can comprise any appropriate network, including an intranet, the Internet, a cellular network, a local area network or any other such network or combination thereof. User devices 1620 can include any network-equipped computing device, for example desktop computers, laptops, smartphones, tablets, e-readers, gaming consoles, and the like. For example, results (e.g., classified images) determined by the multi-aperture imaging system 1605 and the datacube analysis servers 1615 may be sent to designated user devices of patients, doctors, hospital information systems storing electronic patient medical records, and/or centralized health databases (e.g., of the Center for Disease Control) in tissue classification scenarios.

### Example Implementation Outcomes

Background: Morbidity and mortality resulting from burns is a major problem for wounded warfighters and their care providers. The incidence of burns among combat casualties has historically been 5-20% with approximately 20% of these casualties requiring complex burn surgery at the US Army Institute of Surgical Research (ISR) burn center or equivalent. Burn surgery requires specialized training and is therefore provided by ISR staff rather than US Military Hospital staff. The limited number of burn specialists leads to high logistical complexity of providing care to burned soldiers. Therefore, a new objective method of pre-operative and intra-operative detection of burn depth could enable a broader pool of medical staff, including non-ISR personnel, to be enlisted in the care of patients with burn wounds sustained in combat. This augmented pool of care providers could then be leveraged to provide more complex burn care further forward in the role of care of warfighters with burn wounds.

In order to begin addressing this need, a novel cart-based imaging device that uses multispectral imaging (MSI) and artificial intelligence (AI) algorithms to aide in the preoperative determination of burn healing potential has been developed. This device acquires images from a wide area of tissue (e.g., 5.9×7.9 in2) in a short amount of time (e.g., within 6, 5, 4, 3, 2, or 1 second(s)) and does not require the injection of imaging contrast agents. This study based in a civilian population shows that the accuracy of this device in determining burn healing potential exceeds clinical judgement by burn experts (e.g., 70-80%).

Methods: Civilian subjects with various burn severities were imaged within 72 hours of their burn injury and then at several subsequent time points up to 7 days post-burn. True burn severity in each image was determined using either 3-week healing assessments or punch biopsies. The accuracy of the device to identify and differentiate healing and non-healing burn tissue in first, second, and third degree burn injuries was analyzed on a per image pixel basis.

Results: Data were collected from 38 civilian subjects with 58 total burns and 393 images. The AI algorithm achieved 87.5% sensitivity and 90.7% specificity in predicting non-healing burn tissue.

Conclusions: The device and its AI algorithm demonstrated accuracy in determining burn healing potential that exceeds the accuracy of clinical judgement of burn experts. Future work is focused on redesigning the device for portability and evaluating its use in an intra-operative setting. Design changes for portability include reducing the size of the device to a portable system, increasing the field of view, reducing acquisition time to a single snapshot, and evaluating the device for use in an intra-operative setting using a porcine model. These developments have been implemented in a benchtop MSI subsystem that shows equivalency in basic imaging tests.

### Additional Illuminants for Image Registration

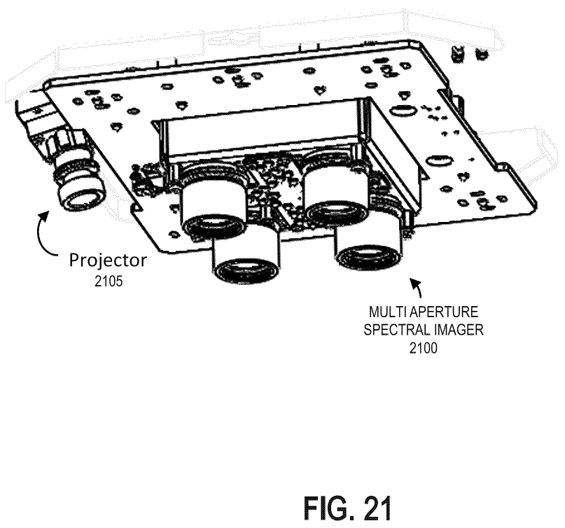

In various embodiments, one or more additional illuminants may be used in conjunction with any of the embodiments disclosed herein in order to improve the accuracy of image registration. FIG. 21 illustrates an example embodiment of a multi aperture spectral imager 2100 including a projector 2105. In some embodiments, the projector 2105 or other suitable illuminant may be, for example, one of the illuminants 1165 described with reference to FIG. 12 above. In embodiments including an additional illuminant such as a projector 2105 for registration, the method may further include an additional exposure. The additional illuminant such as the projector 2105 can project, into the field of view of the imager 2100, one or more points, fringes, grids, random speckle, or any other suitable spatial pattern in a spectral band, multiple spectral bands, or in a broad band, that are individually or cumulatively visible in all cameras of the imager 2100. For example, the projector 2105 may project light of the shared or common channel, broadband illumination, or cumulatively visible illumination that can be used to confirm the accuracy of the registration of the image calculated based on the aforementioned common band approach. As used herein, “cumulatively visible illumination” refers to a plurality of wavelengths selected such that the pattern is transduced by each of the image sensors in the multi-spectral imaging system. For example, cumulatively visible illumination may include a plurality of wavelengths such that every channel transduces at least one of the plurality of wavelengths, even if none of the plurality of wavelengths is common to all channels. In some embodiments, the type of pattern projected by the projector 2105 may be selected based on the number of apertures in which the pattern will be imaged. For example, if the pattern will be seen by only one aperture, the pattern may preferably by relatively dense (e.g., may have a relatively narrow autocorrelation such as on the order of 1-10 pixels, 20 pixels, less than 50 pixels, less than 100 pixels, etc.), while less dense or less narrowly autocorrelated patterns may be useful where the pattern will be imaged by a plurality of apertures. In some embodiments, the additional exposure that is captured with the projected spatial pattern is included in the calculation of disparity in order to improve the accuracy of the registration compared to embodiments without the exposure captured with a projected spatial pattern. In some embodiments, the additional illuminant projects, into the field of view of the imager, fringes in a spectral band, multiple spectral bands, or in a broad band, that are individually or cumulatively visible in all cameras, such as in the shared or common channel, or broadband illumination which can be used to improve the registration of images based on the phase of fringes. In some embodiments, the additional illuminant projects, into the field of view of the imager, a plurality of unique spatial arrangement of dots, grids, and/or speckle in a spectral band, multiple spectral bands, or in a broad band, that are individually or cumulatively visible in all cameras, such as in the shared or common channel, or broadband illumination which can be used to improve the registration of images. In some embodiments, the method further includes an additional sensor with a single aperture or a plurality of apertures, which can detect the shape of the object or objects in the field of view. For example, the sensor may use LIDAR, light field, or ultrasound techniques, to further improve the accuracy of registration of the images using the aforementioned common band approach. This additional sensor may be a single aperture or a multi-aperture sensor, sensitive to light-field information, or it may be sensitive to other signals, such as ultrasound or pulsed lasers.

### Tissue Topological Characterization Using Computer Vision Methods

Topological characterization and/or 3D imaging of tissue structures may provide significant benefits in healthcare and other settings. For example, wound measurements can be an important aspect of wound care and are used for a variety of purposes in the application of healthcare. Wound areas are used to estimate patient outcomes, triage treatment decisions, and estimate the resources required to deliver care. For example, a small third degree burn of approximately 10 cm² in area could be treated with standard wound care depending on its location. Whereas a larger third-degree burn (e.g., 1,000 cm²) would typically be treated with surgical excision and grafting. The area of the burn would be used to estimate the length of the surgical procedure, the amount of tissue to be harvested for grafting, and the reimbursement paid to the surgeon for their services. In the treatment of diabetic foot ulcers, the area and volume of the wound are used to track the progression of the ulcer throughout the treatment process. Furthermore, the geometric contours of a wound are important indicators of the severity and are predictive of healing rates. For example, undermining of the wound edges is identified and measured in each ulcer to determine its full extent into the tissue. Large, undermined areas are indicative of a severe ulcer that will be more difficult to heal. The contours of the wound edges are also critical for the appropriate management of the diabetic foot ulcer. A physician will typically use a scalpel or other sharp instrument to remove unhealthy tissue in and around a chronic wound, such as a diabetic foot ulcer, in a technique called debridement. One of the goals of debridement is to promote the migration of healthy cells into the wound by eliminating, e.g., by cutting, planning, or scraping, contours of the wound having steep edges from the margins of the wound, preferably so that the wound approaches a planar or near planar or flat surface with the skin.

Wound area measurements are routinely performed using a ruler and a probe in the clinical setting. These instruments will be used to measure the wound's length (l), width (w), depth (d), undermining, and/or tunneling. From these basic measurements, the geometry of the wound can be estimated. For example, the area of the wound is typically estimated by modeling the wound as a rectangle such that area=l*w. Similarly, volume can be estimated by modeling the wound as a cuboid such that volume=l*w*d. One could also use an ellipsoid where volume=w*l*d*(π/6). However, wounds do not follow these simple geometric models and therefore currently used techniques for estimating wound area and volume are imprecise and prone to error.

To address these concerns, various tools for measuring the area of the wound have been introduced. These include the calibration of a photo by placing a ruler in the image frame or filling the wound with a putty or paste to create am impression or cast of the wound. Calibrated images annotations made by a user in a software program, such as lines or areas, can be drawn on an image and used to obtain a more accurate measurement of length, width, and area. However, depth cannot be easily estimated using this method. Creating an impression of the wound is an accurate way to measure the wound volume and depth, but cannot be used in a clinical setting because of the risk of contamination or mechanical destruction of the delicate tissues of the healing wound.

Devices have been introduced to simplify the process and provide improvements to these measurement techniques. One device projects a pattern of lasers onto the tissue surface to estimate the tissue measurements and provide an estimation of depth. Another device utilizes the projection of a structured light pattern onto the tissue with a mono-color projector. Another method utilizes 3D photography for the construction of 3D surfaces from a calibrated camera or camera array. There are a variety of other techniques that can be used for estimating real-world 3D geometry from optical sensors that could also be applied for the improvement of wound measurement and geometry, such as LiDAR. For example, LiDAR has been investigated for the detection of tumors in the tissue.

It is important to monitor the change in the physical dimensions of the wound over time. This is informative of the healing of the wound. For example, the typical method of assessing the healing of the wound is to measure Percent Area Reduction (PAR). PAR is equal to the current area of the wound divided by the area of the wound at a previously established baseline multiplied by 100%. A PAR greater than or equal to 50% after 30 days of treatment is associated with a wound that is healing at sufficient rate.

The 3D models created by the present technology can be used to better establish the rate of change of a wound. For example, the current 3D model can be compared to a previous 3D model to report the Percent Volume Reduction (PVR). Also, the absolute change in volume can be better measured using this technique compared to methods that involve the use of rulers and probes.

Herein we describe systems and methods for the assessment of tissue topography for the use in patient management and to guide wound debridement or excision procedures. The method involves the use of one or more images from the target tissue and the reconstruction of the tissue's 3D geometry in a real-world coordinate system with calibrated physical units. We present a case for the technology in a single imager system and a multi-imager system. We demonstrate a method for obtaining local gradients that can be used to investigate the surface geometry of a tissue area and queue a healthcare provider to modifying their treatment protocol.

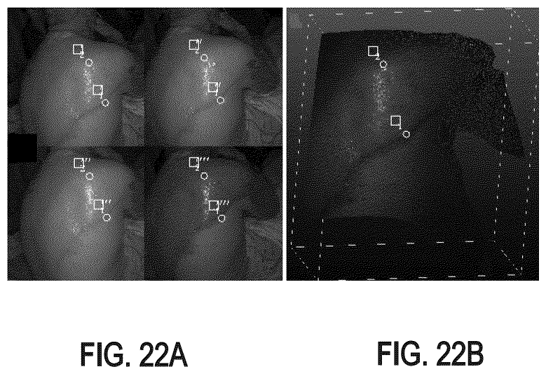

In a single imager system, multiple images (e.g., a video or burst capture) of a target can be acquired and then be aligned to estimate a 3D model of the target as shown, for example, in FIGS. 22A and 22B. First, a set of unique and static image features are identified in each of the images, and depending on some photometric heuristics of each feature, are matched across features. These matches are then used to obtain relative camera poses, assuming pre-calibrated camera lens parameters, or estimated dynamically across the matches. M-estimator Sample Consensus (MSAC) outlier rejection may be used to eliminate false image feature matches at the pose estimation stage.

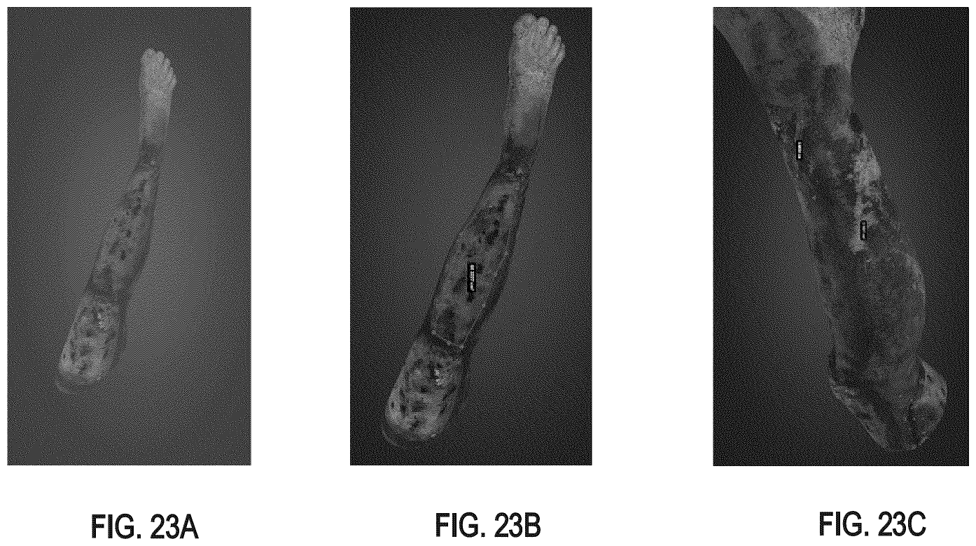

Following sparse localization of the image positions with respect to the target, our method follows a dense multi-view stereo reconstruction, whereby image pairs are selected, stereo rectified and disparity matches are back-projected into a unified 3D model of the target. Such an approach is illustrated in FIGS. 23A, 23B and 23C, which show views of a unified 3D model of a synthetic leg with variations in surface color and texture. Polygons within the images of FIGS. 23B and 23C indicate regations of the surface with their areas annotated, in one example visualization technique in accordance with the present technology. One limitation of this single imager method is that a marker with known dimensions is needed to calibrate the 3D geometry to “real-world” units.

In some embodiments, two or more cameras may be used for topological characterization of a tissue. A set of images from each of at least two image collection devices may be collected from a tissue region (e.g., using any of the multi-aperture imaging systems disclosed herein). All images can be taken simultaneously or in a sequence and then used to estimate a 3D model representation of the tissue. With two or more image collection devices, our method uses geometric constraints to reduce the search space to match every pixel in one image, to those of the images from the other devices. Given the mechanical spacing between the devices, it is possible to triangulate these correspondences into 3D space to create such a model representation. If two or more devices move together to capture multiple frames, the relative motion is estimated with respect to the tissue and 3D projections can be aligned for each position using the estimated sub-models.

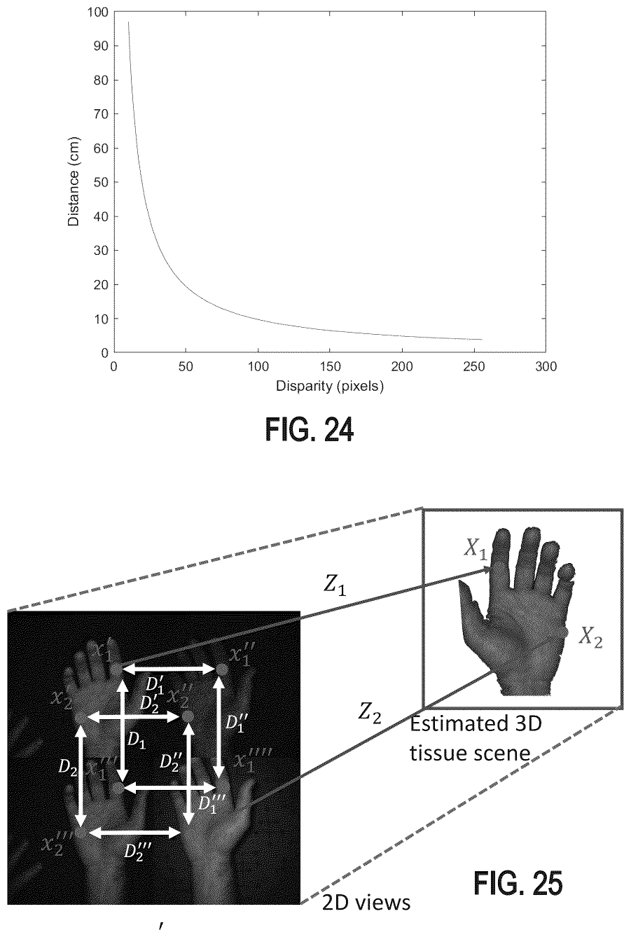

FIG. 24 illustrates an example relationship between pixel disparity and depth or distance information in multi-camera imaging systems. The relationship between pixel disparity and depth or distance can be defined based on the camera focal length and baseline distance between optical centers of the imaging sensors. Accordingly, a depth or distance value for an individual pixel can be equal to the focal length of the camera multiplied by the baseline distance, divided by the determined pixel disparity value for the pixel. In one desirable example implementation, a baseline distance is 55.837 cm in combination with a 6 mm focal length and a 3.45 micrometer pixel pitch, corresponding to a total of 1.74*103 pixels in each image.

Referring now to FIG. 25, image features xᵢ′ features may be observed across multiple cameras at the same time. Assuming ideal cameras, or pre-calibrated intrinsic and extrinsic factors (K, [R|t]), one can estimate the real-world 3D coordinates of such features Xᵢ using solely the pixel disparities between observations Dᵢ, the focal length of the camera f, and the baseline distance.

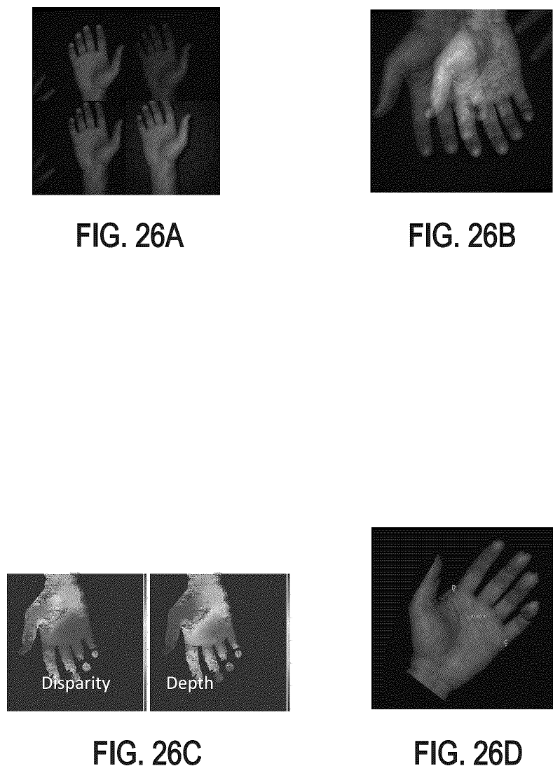

FIGS. 26A-26D illustrate an example process of generating a 3D tissue model based on a plurality of spatially offset 2D images. As shown in FIG. 26A, raw images may be captured by a calibrated camera system.

Referring to FIG. 26B, the images may be rectified to eliminate any optical distortions and/or to compensate for manufacturing imperfections. For example, images may be undistorted and/or rotated. In some embodiments, the rectification operations may result in an epipolar alignment among the images.

Referring to FIG. 26C, two or more rectified frames are used in a dense, multi-view matching process to estimate a per-pixel disparity. In some embodiments, a horopter shift may be implemented prior to determining pixel disparities in order to reduce computation and/or memory requirements. Disparity estimation may include a 1-dimensional per-pixel patch-based similarity estimation using normalized cross correlation and/or Hamming distances from census cost, to estimate the best disparity cost across selected views (e.g., by matching areas constrained to epipolar lines). In some embodiments, the system may choose a minimal fused cost to select a disparity that agrees with all views and constrained geometry. In some embodiments, a mask can be used to match only on pixels considered to be a tissue of interest, as discussed below with regard to image segmentation. Per-pixel disparities may then be mapped to depths based on known extrinsic factors such as baseline distance, to generate a depth map. In some embodiments, disparity estimates can be filtered to improve local surface reconstruction quality. This can be performed on the original disparity maps, or on depth maps or 3D projected points. In the case of a 3D point cloud, per-pixel disparities may be filtered by a Delaunay triangulation fitting a surface to the 3D point cloud.

Referring to FIG. 26D, the depth map can be projected to 3D space using a camera projection model. For example, the depth map can be projected to 3D space using the mathematical relationship between depth, focal length, baseline distance, and disparity discussed above. This projection can result in a 3D point cloud, which in some embodiments can be meshed into a surface. In some embodiments, a reverse back-projection may be used to re-texture the 3D model to output a multi-spectral 3D model. Moreover, in embodiments in which 3 or more image sensors are used, a per-pixel confidence values may be calculated for each individual pixel. A visual representation, such as a confidence map, may be generated based on the determined confidence values. In some embodiments, pixels may be omitted from the 3D model based on, for example, exceeding a threshold confidence margin.

The combined 3D model can be representative of the tissue in multiple, interchangeable forms, including but not limited to: 3D point clouds, 3D textured meshes, 3D parametric surfaces, 3D voxel grids and/or depth heat maps. Such model representations may further associate a statistical accuracy metric, which combines the consistency of model across all view observations.

The 3D tissue model can be calibrated to real-world units. Point samples on the 3D models are related to one another in the physical units of the real-world tissue such that two points with a 1 mm distance in the 3D parametric surface indicate the same 1 mm distance in the real-world tissue. The use of real-world units (e.g., millimeters) allows for derivative analysis (e.g., burn area measurements) to be completed on the device. Any region of tissue may be selected by the user, or by an AI algorithm, and an area of the tissue model may be computed from this region.

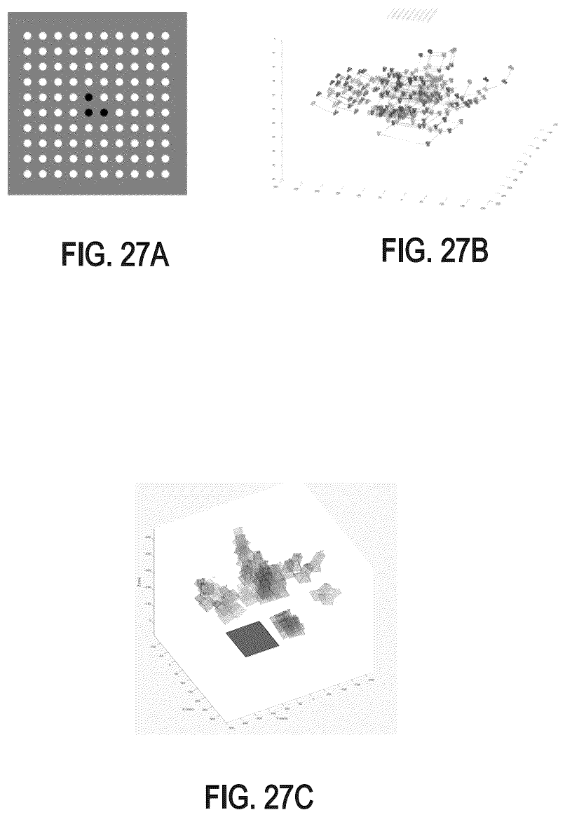

To obtain an accurate real-world, scaled, geometry from the images alone (e.g., without a calibration object in the image with known geometry) the cameras can be pre-calibrated using multi-view geometry methods. The calibration process can be sub-divided into an intrinsic and extrinsic component. If the system includes more than one image collection device, a calibration procedure may implement a standardized calibration target, as shown in FIG. 27A that contains identifiable and unique features, such as keypoints. A set of images can be collected by one or more image collection devices and aggregated into a set of feature correspondence graphs, as shown in FIG. 27B. Parameters and device poses can be simultaneously estimated and optimized to solve for the camera model.

In some embodiments, images of the calibration target may be obtained by a multi-imager system while moving the system around and keeping the target in view of all cameras during the image capture, as shown in FIG. 27C. In some embodiments, a relatively large number of viewpoints can be used to provide optimal calibration. For example, images from the multiple imagers may be taken from 10 or more, 20 or more, 30 or more, 40 or more 50 or more, or a greater number of individual viewpoints. Calibration target features, such as dots at a known geometry, can be detected in all of the obtained calibration images, and may be detected at sub-pixel accuracy to provide improved calibration performance. In some embodiments, such as in the example illustrated in FIG. 27A, the calibration target includes a set of black and white dots to reliably localize the dot centers used for the calibration procedure, and the dots may be extracted by performing Laplacian of Gaussian (LoG) 2^{nd} order gradient filtering, direct-least squares ellipse fitting based on contours, and ordering of the target based on the black dot pattern. Intrinsic factors, such as optical performance of individual imaging sensors and/or lenses, may be estimated based on the individual images. Extrinsic factors, including baseline distance and alignment of optical axes, can further be determined. Overall calibration results can be verified by comparing intrinsic consistency across cameras, similarity of determined extrinsic factors to manufacturing specifications of the multi-imager device, and calibration re-projection errors.

Stability of the calibration may also be tested or monitored using the intrinsic and extrinsic factors. The intrinsic and extrinsic factors relate to the positioning of the components within each camera, therefore if there is motion of a component of a camera (e.g., rotation or translation outside the camera's mechanical tolerance) these parameters may indicate the motion has occurred. Reprojection error, the distal difference between in pixels between a key point detected in a calibrated image and the corresponding world point projected into the same image, may also be useful in determining the stability of the calibration of the camera. Variation in the reprojection error across time may reflect the quality and reusability of the initial calibration outputs. The period for a stability test may, in one example, be between a set of test events (e.g., mechanical shock testing, thermal shock testing, etc.) to simulate normal or extreme use cases of the camera.

The 3D model and images from different locations or imaging devices can be related through a camera projection model, respectively composed of the lens parameters (camera intrinsic parameters) and a 3D rigid-body transformation (extrinsics). The resultant 3D model can therefore be back-projected using such model into 2D image space defined by a virtual camera at some arbitrary location (can be at the same location as a physical camera), or to a synthetic ortho-plane.

Thus, the systems and methods of the present disclosure, calibrated using the calibration processes described herein, can determine one or more measurements (e.g., in a concrete physical dimension such as units of length, area, or volume such as mm, mm², mm³, or other desired length or area unit) of an imaged feature or tissue region (e.g., a depth or maximum depth, an area, a perimeter, a semi-major axis, a semi-minor axis, or other aspect of the tissue region or feature such as a wound, a burn, or the like) without requiring or using a reference object of a known size imaged with the feature or tissue region, as was typically required with existing imaging methods. That is, the calibrated imaging device can capture a set of images of the feature or tissue region and determine the one or more physical measurements of the feature or tissue region based on the images and the depth information obtained from the images, and not based on any type of marker (such as a reference object having a known length or width) that may or may not be shown within the images.

Improvement of calibration or re-calibration may be completed during the capture of tissue samples. An object with known dimensions can be placed into the image set to improve such calibration and the resultant 3D model estimation.

The system can also contain a device for emitting light to the tissue, such as an LED or an LED array to improve the imaging uniformity, as described elsewhere herein.

### Segmentation of Tissue and Tissue Regions

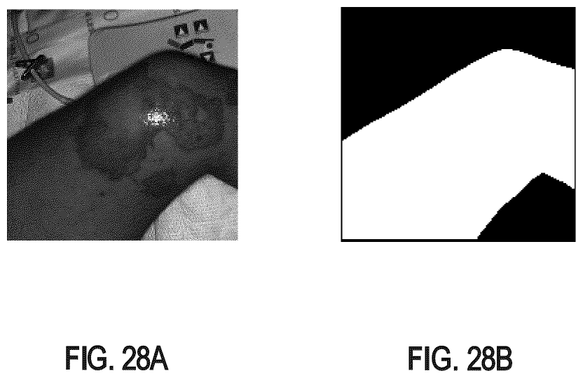

To improve upon the generation of the 3D surface, the tissue contained in each of the images within the set of images can further be segmented from the background. An example of such segmentation is illustrated in FIGS. 28A and 28B. FIG. 28A depicts an image of a burn injury; FIG. 28B depicts a segmentation of the image of FIG. 28A in which the white region corresponds to the region of tissue in the image and the black region corresponds to background pixels. Segmentation can be obtained manually from a person drawing the image, in a semi-automated manner, or by a fully automated method. Semi-automated methods include graph-cuts. Fully automated methods may include simple thresholds applied to the color values in the pixels or ratios of channels. Automated methods may also include supervised and unsupervised machine learning and deep learning methods, such as using convolutional neural networks and the like.

In some embodiments, the generation of the 3D surface can be improved with the use of tissue segmentation by excluding the background regions of the image during the stereo correspondence determination step between two or more imagers. A reduction in the search space may improve several aspects of stereo correspondence. First, by eliminating the background, there is a potential to reduce the probability of incorrectly stereo matched points. In addition, there is also the potential to reduce the number of computations required to complete stereo correspondence calculations due to the reduced image area used for determining correspondence.

The image segmentations can be processed on each image from the multiple image collection devices, and can be aggregated into the joint 3D model. Intersecting segmentation regions in 3D space can be used to calculate to final boundary regions of the 3D model. Example image segmentation algorithms will now be described.

U-Net is an encoder-decoder deep learning semantic segmentation approach, which works with very few training images. The U-Net algorithm uses the skip connection idea to keep the high-resolution features and make better localization.

Unet++ is a deeply-supervised encoder-decoder network where the encoder and decoder sub-networks are connected through a series of nested, dense skip pathways. The encoder extracts features of different spatial resolution which can be used by the decoder to define an accurate segmentation mask.

LinkNet is a fully convolutional neural network for image segmentation. The LinkNet bypasses spatial information from the encoder to the corresponding decoder to improve accuracy along with a significant decrease in processing time. The LinkNet can make efficient use of scarce resources available on embedded platforms.

PSPNet is a fully convolutional neural network for image segmentation, consisting of an encoder and a spatial pyramid which builds on the decoder and does not use fine-features. PSPNet can be used for multi-class segmentation of high-resolution images.

DeepLabV3+ combines a spatial pyramid pooling module and an encoder-decoder structure for image semantic segmentation. It also adds a simple yet effective decoder module to refine the segmentation results especially along object boundaries. The depthwise separable convolution is applied to both atrous spatial pyramid pooling and decoder modules.

A comparison of the performance of the above-listed algorithms in accordance with the tissue image segmentations of the present technology is shown in Table 1 below.

TABLE 1
Intersection over Union values for example image
segmentation algorithms

| | UNet | Unet++ | LinkNet | PSPNet | DeepLabV3+ |
|---|---|---|---|---|---|
| Training IoU | 0.948 | 0.9643 | 0.946 | 0.887 | 0.948 |
| Dev IoU | 0.917 | 0.970 | 0.915 | 0.883 | 0.917 |
| Test Mean IoU | 0.9311 | 0.9427 | 0.9325 | 0.900 | 0.933 |
| Test Std IoU | 0.0649 | 0.0576 | 0.05568 | 0.0944 | 0.053 |
| Test Accuracy | 0.976 | 0.980 | 0.976 | 0.964 | 0.977 |

As shown in Table 1 above, Unet++ produced the highest accuracy in a test setting. Results of different weightings in the Unet++ algorithm are shown in Table 2 below.

TABLE 2
Results of Unet weighting.

| Encoder | ResNet34 | ResNet101 | ResNet152 |
|---|---|---|---|
| Weights | 21M | 42M | 58M |
| Training IoU | 0.964 | 0.965 | 0.957 |
| Dev IoU | 0.927 | 0.934 | 0.933 |
| Test Mean IoU | 0.942 | 0.9526 | 0.9478 |
| Test Std IoU | 0.0576 | 0.0465 | 0.0509 |
| Test Accuracy | 0.980 | 0.983 | 0.981 |

Results of variable amounts of augmentation in the Unet++ algorithm are shown in Table 3 below.

TABLE 3
Results of augmentation in Unet++ algorithm.

| | Less augmentation | More augmentation |
|---|---|---|
| Training IoU | 0.964 | 0.940 |
| Dev IoU | 0.927 | 0.936 |
| Test Mean IoU | 0.942 | 0.9519 |
| Test Std IoU | 0.0576 | 0.04762 |
| Test Accuracy | 0.980 | 0.983 |

Results of different input data set sizes is shown in Table 4 below.

TABLE 4
Results of different input data set sizes in Unet++ algorithm.

| | 320*320*3 | 480*480*3 | 512*512*3 | 1024*1024*3 |
|---|---|---|---|---|
| Training IoU | 0.951 | 0.964 | 0.955 | 0.962 |
| Dev IoU | 0.913 | 0.927 | 0.924 | 0.929 |
| Test Mean IoU | 0.930 | 0.942 | 0.942 | 0.939 |
| Test Std IoU | 0.0650 | 0.0576 | 0.0593 | 0.0652 |
| Test Accuracy | 0.975 | 0.980 | 0.980 | 0.978 |

### Multi-Spectral 3D Imaging

In some embodiments, multi-spectral information can beneficially be obtained and combined with 3D image data. A 3D image with each point represented by more than one spectral channel can be generated from this method in various ways. In one example, a multispectral imaging device can be used as the imager in the system. In this way, the 3D reconstruction would contain the multispectral data that is contained in the initial image set.

In another example, each imager in the array of two more imagers can have a different spectral response (e.g., each camera is sensitive to a different part or parts of the UV/Visible/IR spectrum). From at least a pair of images captured with these imagers, the process of geometric intrinsic calibration, extrinsic calibration, rectification, stereo correspondence, and 3D geometry can be obtained. Stereo correspondence can be obtained using one channel from each of the two or more imagers, through combining all channels of each imager, or sing subsets of channels. Once stereo correspondence is obtained, the 3D geometry can be obtained such that each point in 3D space contains all the channels of all the two or more imagers used in the system. In this way, many more spectral channels can be combined beyond what can be achieved in just one imager.

One way to improve the computation of the stereo correspondence step is to have at least one shared channel across the two imagers (i.e., one channel that shares the same spectral response). For example, two imagers (e.g., each imager corresponding to a combination of an image sensor and a corresponding aperture as described elsewhere herein) may each be configured with a multi-bandpass filter configured to pass a common waveband shared among the two imagers and at least one unique waveband not imaged by the other imager.

### Assessment of Wound Edges

The geometry of the wound edges can be an important factor for predicting the ability of a wound to heal. In wound healing, a critical step is the re-epithelialization of the wound areas. This process occurs when basal keratinocytes travel from the wound edges into the wound's surface by migration and cell division. Basal keratinocytes are the dominant cell type of the epidermis and, once they have migrated into the wound, they promote re-epithelialization by first secreting the basal lamina, an extracellular matrix scaffold. It then takes approximately 2 to 3 weeks for these new cells to form a confluent layer that is keratinized and waterproof.

In wound care, there is much attention paid to the preparation of the wound bed such that it will support this process of re-epithelialization. One important aspect of wound bed preparation is the preparation of the wound edges. For example, edges of the wound that are flat and on the same plane as the surface of the wound bed facilitate the migration of cells needed for re-epithelialization. However, wound edges that are rolled (e.g., epibole) or angled away from the surface of the wound bed impede cell migration. A wound edge that is separated from the wound bed is termed “undermining.” Undermining of the wound edges prevents or inhibits re-epithelialization until the undermined area has been filled with a highly vascularized tissue, called granulation tissue, such that the wound edge is again flat or nearly flat with the wound surface so as to support efficient cell migration.

In the management of wounds, a clinician can manually flatten the edges of the wound to promote cell migration. Flattening of the wound edges is conventionally achieved by cutting or scraping the edges of the wound with a scalpel or brush to a point, which is visually determined by the clinician to be flat or planar to the surface of the skin at the margin of the wound. This process is referred to as debridement. The goal of debridement is to remove epibole, callus, senescent cells, or hypergranulation tissue, as well as flattening the wound. The preparation of wound edges is conventionally performed by a clinician using their skill, experience, and judgment based on eyesight alone.

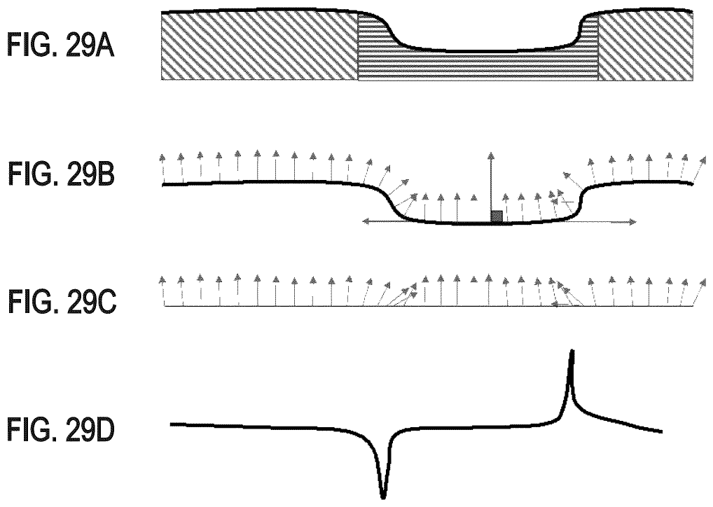

To improve the preparation of wound edges, which also improves wound healing, the 3D topological maps created in accordance with the present technology can be used to assess the geometry of a wound edge before, during, and after debridement. Based on the assessed wound edge geometry, a clinician may be made aware of any unfavorable edge characteristics and use the 3D topological maps to precisely debride the wound and to monitor the debridement while it is being performed, and to assess the efficiency of the debridement (e.g., the efficiency of the flattening of the contours of the wound to reach a planar geometry with the wound margins). One way to achieve this is to compute the gradients of the wound edges with respect to the wound surface, as shown in FIGS. 29A-29D.

FIG. 29A depicts an example 2D cross-section of a wound obtained from 3D image data of a wound as described herein. FIG. 29B illustrates a set of normal vectors to the surface of the tissue calculated at discrete points along the contour of the tissue. In some embodiments, the normal vectors may be isolated and analyzed along a one-dimensional continuum, as shown conceptually in FIG. 29C, and the gradient

$$\frac{d s}{d x}$$

of the wound surface can be calculated as shown in FIG. 29D. Gradients that are larger than a threshold can be used to queue the clinician of an unfavorable edge that would require focused debridement or additional debridement. Additionally, the vectors normal to the surface can be identified, and the gradients can be obtained by comparing these surface normal vectors in local neighborhoods.

### Example Wound Evolution Model

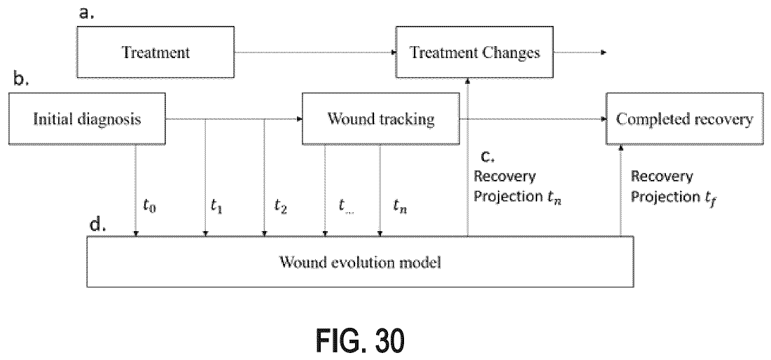

In some methods of the present technology, a wound healing process may be progressively evaluated through a set of photometric, hyperspectral, and geometric observable conditions. FIG. 30 depicts a diagnosis and treatment timeline, to which we introduce a wound evolution model. This model can be progressively augmented as the patient's wound is documented over time with techniques and devices discussed earlier. Such a model may provide feedback to the treatment plan for necessary adjustments, and the model may project a healing rate into the future (e.g., to predict the healing rate of similar wounds in other patients, which optionally, may have similar health care metrics such as peripheral vascular disease, diabetes, insulin resistance, or obesity).

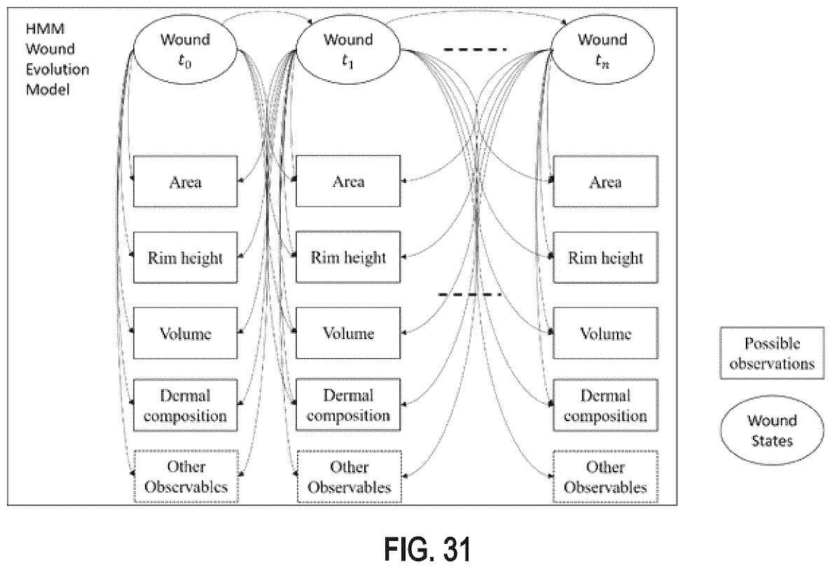

As shown in FIG. 31, the wound evolution model can be represented as a Hidden Markov Model (HMM) whereby a set of measurable observables collected through the image device are representations of non-observable wound states. For example, a sub-dermal infection state of the wound may not be directly known but estimated by the model through the present and the past imaging observations. The model may form connections across some or all observed timesteps. Furthermore, each edge can be dynamically adjusted with new data availability to better model the changes of the wound. Example causal and observable relationships between a set of wound states and some proposed observables measured manually, semi-automatically or fully-automatically, with external methods, or imaging methods proposed earlier, are illustrated in FIG. 31.

Through the measurements obtained by these methods, the device could provide the user with information that is required to inform on the severity of the wound and the services required to treat the wound. Where the wound is a burn, the area of the burn may be computed from the 3D surface using the widely accepted formula for calculating body surface area (BSA) of BSA_{cm}_{²}=C (weight_{kg}^{a}) (height_{cm}^{b}). Where C, a, and b are empirical constants which may, for example, be estimated as 94.9, 0.441, and 0.655. These constants may be adjusted as accepted values in the field are adjusted. The percent total body surface area affected by the burn (% TBSA) may then be calculated using the formula % TBSA=100.0 (burn_{cm}_{²}/BSA_{cm}_{²}).

This may be especially useful for the purposes of generating a report used for billing the patient or the patient's insurance provider. For example, in the US physicians are reimbursed for the care of small burns differently than larger burns (e.g., CPT Code 16025 is for a medium size burn such as the whole face or whole extremity, or 5% to 10% of total body surface area. Whereas CPT Code 16030 is for large burns of more than one extremity, or greater than 10% of total body surface area.). In another example, the machine could measure a burn's area in cm² and then recommend the appropriate billing code for a subsequent skin grafting procedure. In this example, CP 15002 is used for the excision of a burn and the creation of the burn site by for a skin graft up to the first 100 cm² or 1% of body area of infants and children. Additional CPT codes, +15003 CPT and +15005 CPT, are then applied for additional areas in increments of 100 cm².

### Example Diabetic Foot Ulcer 3D Characterization

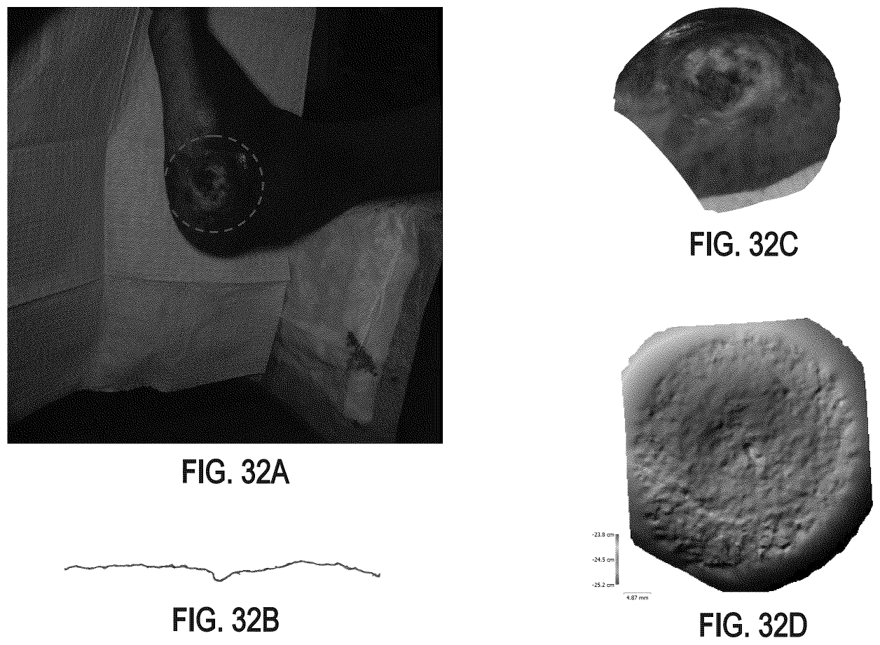

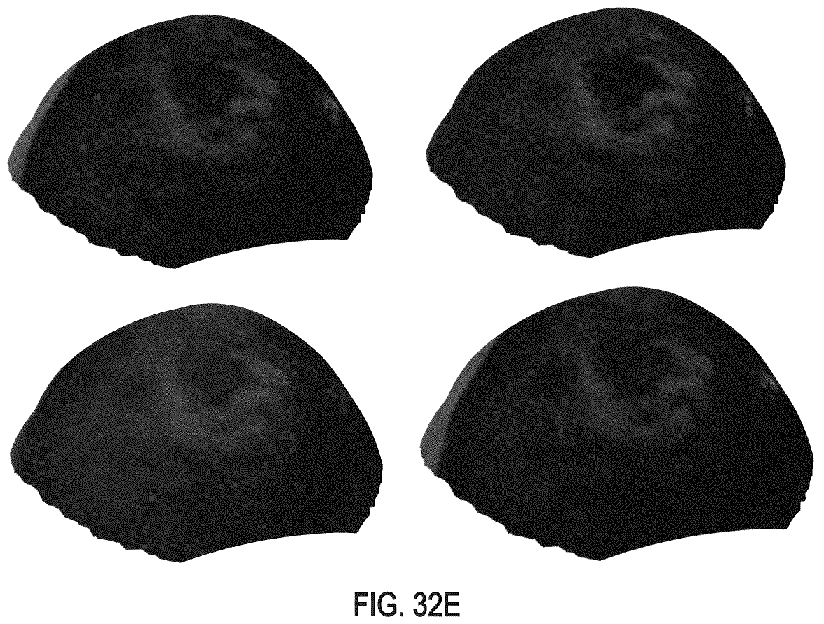

FIGS. 32A-32E illustrate an example 3D characterization of a diabetic foot ulcer in accordance with the present technology. FIG. 32A is a photograph of a foot having a diabetic foot ulcer thereon, corresponding to a region of interest encircled in a dashed line. FIG. 32B depicts a 2D cross-section taken along a straight line across the region of interest and indicating the topology of the diabetic foot ulcer. FIG. 32C illustrates a visual representation of 3D multi-spectral image data representing the region of interest in the image of FIG. 32A. FIG. 32D illustrates a heat map showing per-pixel depth information corresponding to the region of interest in the image of FIG. 32A. FIG. 32E illustrates example multi-spectral re-texturing of the estimated 3D surfaces of the tissue in the region of interest. In the re-textured images of FIG. 32E, original image pixels are aligned into the generated 3D model using the calculated geometry of the diabetic foot ulcer.

Example Application for Classification, Detection, and/or Diagnosis of Cancerous Lesions

The use of 3D geometry and tissue topology with or without a combination of reflectance measurements, as described herein, can be applied to detection, classification, and/or diagnosis of cancerous tissue. For example, in melanoma, doctors make a primary assessment of the probability of a lesion or mole to be or cancerous or transition to cancerous tissue. This primary assessment is typically based on clinician judgment on the visual inspection of the lesion using a handheld instrument called a dermatoscope. Dermatoscopic assessment has been shown to be only 65% accurate and relies on the experience of the clinician. The features to be concerned with when trying to detect melanoma are asymmetry, border, color, diameter, and evolution. These features are known as the “ABCDE's of Melanoma.”

Different types of melanoma have different appearances. The common superficial spreading melanoma has the appearance of a brown and black stain spreading from a mole. Over time it grows slowly outward slowly before invading deeper layers of skin. Nodular melanoma grows vertically as dome-shaped bumps with blue to black in color. Lentigo maligna melanoma looks like a large, tan stain with an uneven border that is flat and then later bumpy. Acral lentiginous melanoma appears on palms, soles, knuckles, elbows, and knees. Subungual melanoma a subtype of acral lentiginous melanoma develops on finger nails and toc nails. This melanoma looks like a bruise, or in rare cases, it is colorless (amelanotic or unpigmented). Amelanotic melanoma has no pigmentation, and looks like a reddish patch or skin-colored lump. Are circilar or ovoid with regular borders

Computation methods for the assessment of the ABCDE method have been developed. These methods are applied to 2D optical images obtained from images of skin lesions. The methods of producing a 3D mesh of the mole's topology have been applied to further improve the quantification of the ABCDE parameters of the mole that is suspect for melanoma.

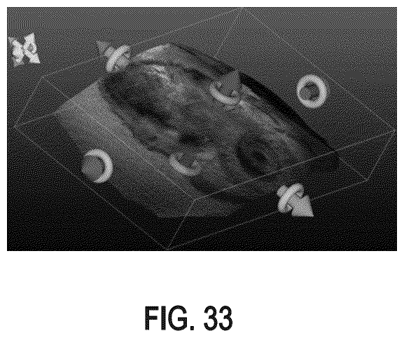

A desirable feature of the methods described in this application are two-fold. First, the generation of the 3D image of the lesion, mole, discolored skin, or wound inherently contains the mapping of tissue reflectance (color) into each point in the 3D mesh as shown, for example, in FIG. 33. Using the generated 3D image also allows for equalization of the reflectance values, such that the effect of the inverse square law on the perceived intensity of light may be reduced, improving the ability to assess the wound. Prior methods required co-localizing a 2D color image onto a 3D tissue map, resulting in mapping errors and providing only an estimation of color at each point in 3D space. Second, the methods described herein allow the generation of 3D geometry at size scales that are determined by the optics of the lenses. Therefore, microscopic images of lesion morphology can be obtained in 3D if there is sufficient resolution of the lesion as determined by the lens parameters on the sensor or sensor array.

### Terminology

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

The disclosed processes may begin in response to an event, such as on a predetermined or dynamically determined schedule, on demand when initiated by a user or system administer, or in response to some other event. When the process is initiated, a set of executable program instructions stored on one or more non-transitory computer-readable media (e.g., hard drive, flash memory, removable media, etc.) may be loaded into memory (e.g., RAM) of a server or other computing device. The executable instructions may then be executed by a hardware-based computer processor of the computing device. In some embodiments, the process or portions thereof may be implemented on multiple computing devices and/or multiple processors, serially or in parallel.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the scope of the disclosure. As can be recognized, certain embodiments described herein can be embodied within a form that does not provide all of the features and benefits set forth herein, as some features can be used or practiced separately from others. All changes which come within the meaning and range of equivalency of the claims are to be embraced within their scope.

What is claimed is:

1. An imaging system comprising:
a plurality of image sensors configured to receive light reflected by a tissue region in at least a first predetermined waveband;
a memory storing computer-executable instructions; and
one or more processors configured by the computer-executable instructions to at least:
cause each of the at least two image sensors to capture an image of the tissue region;
identify corresponding sets of pixels in the captured images, each set comprising a pixel of a first image captured by a first image sensor of the plurality of image sensors and a pixel of a second image captured by a second image sensor of the plurality of image sensors;
determine pixel disparities for the pixels of the corresponding sets of pixels, preferably groups of pixel disparities or individual pixel disparities;
determine, based at least in part on the pixel disparities and a baseline distance between the first and second image sensors, distance values associated with the individual corresponding sets of pixels; and
generate a three-dimensional (3D) model of the tissue region based at least in part on the distance values.

2. The imaging system of claim 1, wherein the 3D model of the tissue region comprises at least one of a 3D point cloud, a 3D textured mesh, a 3D parametric surface, or a 3D voxel grid.

3. The imaging system of claim 1, wherein the instructions further configure the one or more processors to determine one or more measurements of at least a portion of the tissue region based at least in part on the 3D model and on a prior calibration of the imaging system.

4. The imaging system of claim 3, wherein the one or more measurements comprise at least one of a length measurement, an area measurement, or a volume measurement of a portion of the tissue region.

5. The imaging system of claim 3, wherein the one or more measurements comprise a cross-sectional profile of a portion of the tissue region.

6. The imaging system of claim 3, wherein the prior calibration is performed based on imaging a standardized calibration target with the imaging system.

7. The imaging system of claim 3, wherein the one or more measurements are not determined based on a marker placed within a field of view of the imaging system.

8. The imaging system of claim 3, wherein the instructions further configure the one or more processors to perform one or more diagnostic analyses based at least in part on the one or more measurements.

9. The imaging system of claim 3, wherein the instructions further configure the one or more processors to store a medical billing code identified based at least in part on the one or more measurements.

10. The imaging system of claim 1, wherein the instructions further configure the one or more processors to segment the captured images prior to identifying the corresponding sets of pixels.

11. The imaging system of claim 10, wherein the segmenting comprises identifying at least background pixels and tissue region pixels.

12. The imaging system of claim 11, wherein the background pixels are excluded from the identification of corresponding sets of pixels.

13. The imaging system of claim 1, further comprising at least one light source configured to illuminate the tissue region with light of at least the first predetermined waveband.

14. The imaging system of claim 1, wherein each image sensor of the plurality of image sensors is configured to image the tissue region in at least the first predetermined waveband and a second waveband different from the second waveband of at least one other image sensor of the plurality of image sensors.

15. The imaging system of claim 14, wherein the instructions further configure the one or more processors to obtain multispectral image data associated with the tissue region based on the captured images.

16. The imaging system of claim 15, wherein the instructions further configure the one or more processors to align the multispectral image data based on image data corresponding to the first predetermined waveband.

17. The imaging system of any claim 1, wherein the tissue region includes at least a portion of a wound, an uneven tissue surface, hyperpigmentation, a lesion, such as a cancerous lesion including, but not limited to, a squamous cell carcinoma, basal cell carcinoma, Merkel cell carcinoma, melanoma, or an actinic keratosis.

18. The imaging system of claim 17, wherein the wound comprises at least one of a burn, a surgical wound, a pressure ulcer, or a diabetic foot ulcer.

19. The imaging system of claim 1, wherein the instructions further configure the one or more processors to detect at least a portion of an edge of the wound, uneven tissue surface, hyperpigmentation, or lesion based on the 3D model.

20. The imaging system of claim 19, wherein the instructions further configure the one or more processors to compute one or more gradients of the edges of the wound, uneven tissue surface, hyperpigmentation, or lesion with respect to a surface of the wound, uneven tissue surface, hyperpigmentation, or lesion.

21. The imaging system of claim 20, wherein the instructions further configure the one or more processors to determine a status of the wound, uneven tissue surface, hyperpigmentation, or lesion based on a comparison of the one or more gradients to a predetermined gradient threshold.

22. The imaging system of claim 21, wherein the instructions further configure the one or more processors to evaluate a change in the status of the wound, uneven tissue surface, hyperpigmentation, or lesion based on a plurality of 3D models generated by the imaging system at different times.

23. The imaging system of claim 19, wherein the instructions further configure the one or more processors to determine a characteristic comprising at least one of an area, a rim height, a volume, or a dermal composition of the wound, uneven tissue surface, hyperpigmentation, or lesion based on the 3D model and on the detected at least a portion of an edge of the wound, uneven tissue surface, hyperpigmentation, or lesion.

24. The imaging system of claim 23, wherein the instructions further configure the one or more processors to evaluate a change in the characteristic based on a plurality of 3D models generated by the imaging system at different times.

25. The imaging system of claim 1, wherein the instructions further configure the one or more processors to perform image rectification or transformation prior to identifying the corresponding sets of pixels.

26. The imaging system of claim 25, wherein the image rectification or transformation comprises correcting at least one of scale, distortion, skew, perspective, or rotation of the captured images.

27. The imaging system of claim 1, wherein the instructions further configure the one or more processors to output a visual representation of the tissue region based on the 3D model of the tissue region.

28. The imaging system of claim 27, wherein the visual representation comprises two-dimensional image data from at least one of the captured images fitted to the 3D model.

29. The imaging system of claim 27, wherein the visual representation comprises a heatmap.

30. The imaging system of claim 27, wherein the visual representation comprises a two-dimensional cross-sectional profile of at least a portion of the tissue region.

31. The imaging system of claim 1, wherein the plurality of image sensors include at least three image sensors, and wherein each set further comprises a pixel of a third image captured by a third image sensor of the plurality of image sensors.

32. The imaging system of claim 31, wherein the computer-executable instructions further configure the one or more processors to determine confidence values corresponding to the distance values associated with the individual corresponding sets of pixels.

33. The imaging system of claim 32, wherein, for the individual corresponding sets of pixels, the distance value is determined based on the pixels of the first image and the second image and the confidence value is determined based at least in part on the pixels of the third image.

34. The imaging system of claim 32, wherein the confidence value comprises an uncertainty value expressed in a unit of dimension, such as length.

35. The imaging system of claim 32, wherein the computer-executable instructions further configure the one or more processors to generate a confidence map of the tissue region based on the 3D model and the determined confidence values.

36. The imaging system of claim 32, wherein the computer-executable instructions further configure the one or more processors to exclude from the 3D model pixels having a confidence value satisfying a predetermined threshold.

37. The imaging system of claim 32, wherein identifying the corresponding sets of pixels in the captured images comprises evaluating a cost function to identify matching areas or pixels between the first image and the second image.

38. The imaging system of claim 37, wherein identifying the matching areas or pixels comprises minimizing the cost function.

39. Use of the imaging system of claim 1 in a wound debridement procedure, optionally before, during or after a wound debridement procedure such as flattening the contour or edge of a wound.

40. A method of improving the healing of a wound comprising generating a 3D model of the wound using an imaging system of claim 1 and debriding the wound with reference to the 3D model generated by the imaging system such as by referring to the 3D model of the wound prior to, during, or after a debridement procedure e.g., flattening the contour or edge of the wound.
